(19) United States
(12) Patent Application Publication
(10) Pub. No.: US 2009/0042186 A1
(43) Pub. Date: Feb. 12, 2009

(54) MAPPING NEW SITES FOR ANTIBIOTIC ACTION IN THE RIBOSOME

(76) Inventors: Alexander Mankin, River Forest, IL (US); Aymen Samir Yassin, Cairo (EG)

Correspondence Address: POLSINELLI SHALTON FLANIGAN SUELTHAUS PC, 700 W. 47TH STREET, SUITE 1000, KANSAS CITY, MO, 64112-1802, US

(21) Appl. No.: 11/913,756

(22) PCT Filed: May 4, 2006
(86) PCT No.: PCT/US06/16790

§ 371 Date: Aug. 22, 2008

Related U.S. Application Data
(60) Provisional application No. 60/678,444.
(60) Provisional application No. 60/750,508, filed on Dec. 15, 2005.

(51) Int. Cl. C12Q 1/68; C07H 21/00

(52) U.S. Cl. 435/6; 536/23.1

(57) ABSTRACT

The present invention provides a method of mapping and identifying new sites for antibiotic action in the ribosome of a microorganism. The method comprises: (a) providing a random mutant library of the rRNA genes of the microorganism prepared by randomly mutating the rRNA genes of the microorganism; (b) enriching the library in clones with deleterious rRNA mutants by negative selection; (c) screening for clones with deleterious rRNA mutations; (d) mapping the deleterious rRNA mutations in the clones obtained from step (c) to identify sites in the rRNA which are important functional sites in the ribosome; and (e) selecting functional sites identified in the rRNA in step (d) which are not targeted by a known antibiotic as new sites for antibiotic action for the microorganism. The rRNA gene can be the 16S rRNA of the small subunit or the 23S rRNA or the 5S rRNA of the large subunit of the ribosome of the microorganism.

### CROSS-REFERENCE TO RELATED APPLICATION

This application claims the priority of Unites States provisional application Ser. Nos. 60/678,444 filed May 6, 2005, and 60/750,508 filed Dec. 15, 2005, which are incorporated herein by reference and made a part hereof.

### FEDERALLY SPONSORED RESEARCH OR DEVELOPMENT

This invention was made with United States government support under National Institutes of Health grant U19 A156575 (A.S.M.). The United States government has certain rights in the invention.

### BACKGROUND OF THE INVENTION

1. Field of the Invention

The present invention is related generally to the mapping of new sites for antibiotic action in both the small and large subunits of the ribosome of a microorganism.

2. Background of the Invention

The ribosome is the central component of the protein synthesis apparatus of the cell. Ribosomes are composed of two subunits, the small subunit and the large subunit. Each subunit contains ribosomal RNA (rRNA) and proteins. rRNA represents the major structural and functional component of the ribosome. In prokaryotes, the small subunit contains a 16S (Svedberg units)ᵣRNA and the large subunit contains a 23S rRNA and a 5S rRNA.

With a molecular weight of about 2.5 million Daltons and more than 50 different RNA and protein building blocks, the ribosome represents one of the largest and most complex enzymes in the cell. rRNA accounts for two thirds of the ribosome and is responsible for its main functions in protein synthesis: interpretation of genetic information and polymerization of amino acids into a polypeptide. rRNA is also intimately involved in known auxiliary activities of the ribosome, such as nascent peptide release, binding of translation factors, GTP hydrolysis, etc. (Ramakrishnan, V. (2002) Cell 108, 557-572).

The ribosome is the predominant antibiotic target in the bacterial cell. A large variety of natural and synthetic antibiotics interfere with translation by binding to rRNA and preventing correct placement of ribosomal ligands, corrupting rRNA structure, or affecting conformational flexibility of rRNA (Cundliffe, E. (1990) in The Ribosome: Structure, Function, & Evolution, eds. Hill, W. E., Dahlberg, A., Garrett, R. A., Moore, P. B., Schlessinger, D. & Warner, J. R. (American Society for Microbiology, Washington, D.C.), pp. 479-490). Advantages of the ribosome as an antibiotic target stems from its RNA-based design. The multiplicity of rRNA genes in microbial genomes makes it difficult for a microorganism to develop resistance by mutating the drug-binding site (Sigmund, C. D., Ettayebi, M., Borden, A. & Morgan, E. A. (1988) Methods Enzymol. 164, 673-690). Furthermore, RNA offers fewer mutational options than protein enzymes (3 vs 19, respectively), which makes it more difficult for a microbial pathogen to “find” a mutation that would reduce antibiotic binding without compromising functional integrity of the enzyme. In the clinical setting, resistance to protein synthesis inhibitors is usually associated with the acquisition of resistance genes (often originating in the antibiotic producers) rather than mutation of target sites (Farrell, D. J., Douthwaite, S., Morrissey, I., Bakker, S., Poehlsgaard, J., Jakobsen, L. & Felmingham, D. (2003) Antimicrob. Agents Chemother. 47, 1777-1783; Wright, G. D. (2003) Curr. Opin. Chem. Biol. 7, 563-569), illustrating the high cost of fitness and the difficulty of acquiring and fixing rRNA mutations.

Known ribosomal inhibitors act on a fairly limited number of sites usually located within the ribosomal functional centers. Mapping the sites of the drug action has played an important role in the identification and characterization of functionally critical regions of the ribosome (Cundliffe, E. (1987) Biochimie 69, 863-869; Garrett, R. A. & Rodriguez-Fonseca, C. (1996) in Ribosomal RATA: Structure, Evolution, Processing, and Function in Protein Biosynthesis, eds. Zimmermann, R. A. & Dahlberg, A. E. (CRC Press, Boca Raton), pp. 327-355). However, given the enormous size and functional complexity of the ribosome, the number of possible antibiotic targets and sites of functional importance is likely to exceed those currently known. Occasional serendipitous discovery of antibiotics acting on novel ribosomal sites and subsequent recognition of the functional significance of such sites supports this notion (Adrian, P. V., Mendrick, C., Loebenberg, D., McNicholas, P., Shaw, K. J., Klugman, K. P., Hare, R. S. & Black, T. A. (2000) Antimicrob. Agents Chemother. 44, 3101-3106; Belova, L., Tenson, T., Xiong, L., McNicholas, P. M. & Mankin, A. S. (2001) Proc. Natl. Acad. Sci. USA 98, 3726-31). Nonetheless, only a few attempts have been made to identify new sites of antibiotic action and to understand the activity of the associated centers in the ribosome (Laios, E., Waddington, M., Saraiya, A. A., Baker, K. A., O'Connor, E., Pamarathy, D. & Cunningham, P. R. (2004) Arch. Pathol. Lab. Med. 128, 1351-1359).

The performance of the rRNA sites critical for ribosome functions, structure or assembly critically depends on their chemical makeup. Therefore, nucleotide alterations at such sites are expected to be deleterious for the cell. The distribution of the recognized ribosomal functional sites and the sites of antibiotic action clearly correlate with the location of the known deleterious mutations in rRNA (Triman, K. L. & Adams, B. J. (1997) Nucleic Acids Res. 25, 188-191). Thus, deleterious mutations in rRNA serve as hallmarks of both functionally important ribosomal centers and antibiotic sites. In a search for new putative functional sites in the ribosome which can be targeted by new antibiotics, we used a combination of random mutagenesis and negative selection to identify a variety of deleterious mutations in the rRNA of small and large ribosomal subunits of bacterial ribosomes. As the result of these experiments, we isolated a collection of mutations in rRNA that highlight structure-sensitive rRNA centers that can be targeted by new antibiotics, including rRNA regions with previously unrecognized functional significance.

Several references have disclosed the targeting of rRNA for new antibiotic development. For example, U.S. Pat. Nos. 6,947,844, 6,947,845 and 6,952,650 to T. Steitz et al. disclose a method to provide high resolution X-ray structures of the large ribosomal subunit for identifying ribosome-related ligands and methods for designing ligands with specific ribosome-binding properties as well as ligands that may act as protein synthesis inhibitors.

United States Patent Application No. 2004/0137011 by P. R. Cunningham et el. discloses methods to develop antibiotics that are not susceptible to the development of antibiotic resistance by screening for antibiotics that bind to mutant ribosomes.

International Patent Application WO 00/32619 by C. S. Dammel et al. discloses a method for screening a test compound as a potential antibiotic by its ability to interfere with the assembly of the ribosome by interfering with the binding of the ribosomal proteins to the rRNA.

Z. Ma et al. disclose in United States Patent Application No. US2005/0118624 a method for identifying compounds that bind to specific ribosome sites that are known targets of antibiotic action.

E. E. Swayze et al. disclose in United States Patent Application No. US2003/0187258 a method to identify specific classes of compounds that act upon known antibiotic sites used by aminoglycoside antibiotics.

M. Afshar et al. disclose in United States Patent Application No. US2002/0138240 computational methods in docking and in silico screening to identify binding of compounds to identified sites in a biological macromolecule such as the RNA.

H. D. Robertson et al. disclose in United States Patent Application No. US2003/0143247 a method to identify compounds which prevent binding of the eukaryotic ribosome to the viral internal ribosome entry site (IRES).

U.S. Pat. No. 5,958,695 to A. Mankin discloses a method of screening to find new antibiotics binding to a specific site in the ribosome.

These and other aspects and attributes of the present invention will be discussed with reference to the following drawings and accompanying specification.

### BRIEF SUMMARY OF THE INVENTION

An embodiment of the present invention provides a method of mapping and identifying new sites for antibiotic action in the ribosome of a microorganism. The method comprises: (a) providing a random mutant library of the rRNA genes of the microorganism prepared by randomly mutating the rRNA genes of the microorganism; (b) enriching the library in clones with deleterious rRNA mutants by negative selection; (c) screening for clones with deleterious rRNA mutations; (d) mapping the deleterious rRNA mutations in the clones obtained from step (c) to identify sites in the rRNA which are important functional sites in the ribosome; and (e) selecting functional sites identified in the rRNA in step (d) which are not targeted by a known antibiotic as new sites for antibiotic action for the microorganism. The rRNA gene can be the 16S rRNA of the small subunit or the 23S rRNA or the 5S rRNA of the large subunit of the ribosome of the microorganism.

Another embodiment of the present invention discloses new functional sites in the rRNA of the ribosome of a microorganism identified by the methods of the present invention. These functional sites represent sites for new antibiotic action in the ribosome of the microorganism.

Yet another embodiment of the present invention discloses a method of screening for new antibiotics by identifying molecules that bind to the new functional ribosomal sites identified by the methods of the present invention to interfere with growth of the microorganism.

In a further embodiment, the present invention discloses a ribosomal site for new antibiotic action for a microorganism. A mutation in the site results in interfering with growth of the microorganism, and the site is not a known target of a known antibiotic.

In yet a further embodiment, the present invention discloses a new antibiotic for a microorganism. The new antibiotic binds to a ribosomal site of the microorganism to interfere with growth of the microorganism wherein a mutation in the ribosomal site results in interfering with the growth of the microorganism and the site is not a known target of a known antibiotic.

### BRIEF DESCRIPTION OF THE DRAWINGS

FIG. 1 is a schematic diagram showing dominant deleterious mutations in Escherichia coli 16S rRNA selected from a random mutant library. Mutant positions are colored according to the severity of the phenotype: black, strongly deleterious; light gray, moderately deleterious; and dark gray, mildly deleterious. Single nucleotide deletions within a stretch of identical nucleotides are marked by asterisks. The relevant helices of 16S rRNA are marked by their numbers according to (31). The 16S rRNA secondary structure (38) was retrieved from the Web site http://www.rna.icmb.utexas.edu/(26) and simplified for clarity of presentation;

FIG. 2 shows the results of protein synthesis activity of individual 16S rRNA mutants in the specialized ribosome system. Activity of the LacZ reporter (Miller units) in cells expressing 16S rRNA that carried altered anti-Shine-Dalgarno region but no other mutations (“WT”) was taken as 100%. Bars representing protein synthesis activity of individual mutants are colored according to the scheme used in FIG. 1: black, strongly deleterious; light gray, moderately deleterious; and dark gray, mildly deleterious;

FIG. 3 shows the sucrose gradient profiles of polysomes prepared from cells transformed with wild type or mutant pLK45 plasmids. Positions of 30S subunits, 50S subunits, and 70S ribosomes are indicated by arrowheads. The severity of deleterious phenotype conferred by the mutation (strong, moderate or mild) is indicated;

FIG. 4 is a schematic diagram showing mutations in the sites of action of known antibiotics (black) and sites not targeted by the studied drugs (gray). Mutations were attributed to the antibiotic binding sites if they were located within 6 Å from the site of the binding of antibiotic to the Thermus aquaticus 30S ribosomal subunit in the crystalline state;

FIG. 5 is a schematic diagram showing the distribution of the 16S rRNA mutations between the known functional sites (dark gray) and sites of less recognized functional significance (intermediate gray). A mutation is considered to belong to a functional site if it is a part of an rRNA element involved in the corresponding function of the ribosome. Also shown are previously identified deleterious mutations (Triman, K. L. & Adams, B. J. (1997) Nucleic Acids Res. 25, 188-191): light gray if absent and asterisks if present among mutations identified in the random mutant library;

FIG. 6 shows the cluster of deleterious mutations in the 16S rRNA site comprising elements of helices 5 and 15. Positions are colored according to the scheme used in FIG. 1. (A) The backbone diagram of 16S rRNA. (B) Space-fill model of T. thermophilus 30S subunit (Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339) showing 16S rRNA in light gray and ribosomal proteins in dark gray;

FIG. 7 shows the cluster of deleterious mutations in helix 24. Positions are colored according to the scheme used in FIG. 1. (A) The backbone diagram of 16S rRNA. (B) Space-fill model of T. thermophilus 30S subunit (Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339) showing 16S rRNA in light gray and ribosomal proteins in dark gray;

FIG. 8 shows the cluster of deleterious mutations in the site comprising elements of helices 12 and 21. Positions are colored according to the scheme used in FIG. 1. (A) The backbone diagram of 16S rRNA. (B) Space-fill model of T. thermophilus 30S subunit (Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339) showing 16S rRNA in light gray and ribosomal proteins in dark gray;

FIG. 9 shows the cluster of deleterious mutations in the site comprising elements of helices 35-37. Positions are colored according to the scheme used in FIG. 1. (A) The backbone diagram of 16S rRNA. (B) Model of T. thermophilus 30S subunit (Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339) showing 16S rRNA in space-fill representation and ribosomal proteins in ribbon representation;

FIG. 10 is a schematic diagram showing the length of the 23S and 5S rRNA gene segments in segment-libraries in base pairs and the restriction sites flanking each;

FIG. 11A is a schematic diagram showing positions of mutations identified in the 5′ half end of the 23S rRNA; FIG. 11B shows the positions of mutations identified in the 3′ end half of the 23S rRNA. Single- or double-nucleotide deletions within a stretch of identical nucleotides are marked by an asterisk;

FIG. 12A is a schematic diagram showing the secondary structure of the 5′ end of 23S rRNA showing the classification of the mutations according to the severity of the phenotype. Black indicates the strong deleterious mutations; light gray, the intermediate deleterious mutations; and dark gray, the mild deleterious mutations. Single- or double-nucleotide deletions within a stretch of identical nucleotides are marked by an asterisk; FIG. 12B shows the secondary structure of the 3′ end of 23S rRNA showing the classification of the mutations according to the severity of the phenotype. Black indicates the strong deleterious mutations; light gray, the intermediate deleterious mutations; and dark gray, the mild deleterious mutations, Single-nucleotide deletions within a stretch of identical nucleotides are marked by an asterisk;

FIG. 13 is a schematic diagram showing the secondary structure of the 3′ half of the 23S rRNA indicating the positions of identified deleterious mutations located close to the known sites of antibiotic action;

FIG. 14A is a schematic diagram showing the mutations in the 5′ half of the 23S rRNA located in the known functional site (dark gray) or in sites with unrecognized functional importance (light gray); FIG. 14B shows the mutations in the 3′ half of the 23S rRNA located in the known functional site (dark gray) or in sites with unrecognized functional importance (light gray);

FIG. 15 is a schematic diagram showing the distribution of previously identified dominant lethal mutations relative to the newly mapped deleterious mutations in the 3′ half of 23S rRNA;

FIG. 16 shows the cluster of deleterious mutations between domains II and V of the 23S rRNA (cluster V). (A) Secondary structure of the 23S rRNA showing the locations of the mutations. (B) The cluster shown on a backbone model of the D. radiodurans 50S subunit (interface view). The positions of the mutations are colored in dark gray or light gray. The rest of the rRNA is in gray. The ribosomal proteins are omitted for clarity;

FIG. 17 shows the Cluster VI of deleterious mutations in domain I of the 23S rRNA. (A) Secondary structure of the 23S rRNA showing the locations of the mutations. (B) The cluster shown on a backbone model of the D. radiodurans 50S subunit (solvent side view). The positions of the mutations are colored in intermediate gray. The rest of the rRNA is in gray. The ribosomal proteins are omitted for clarity;

FIG. 18 shows the Cluster VII of deleterious mutations in domain III of the 23S rRNA. (A) Secondary structure of the 23S rRNA showing the locations of the mutations. (B) The cluster shown on a backbone model of the D. radiodurans 50S subunit (interface view). The positions of the mutations are colored in light gray and intermediate gray. The rest of the rRNA is in gray. The ribosomal proteins are omitted for clarity;

FIG. 19 shows the Cluster VIII of deleterious mutations in domain III of the 23S rRNA. (A) Secondary structure of the 23S rRNA showing the locations of the mutations. (B) The cluster shown on a backbone model of the D. radiodurans 50S subunit (solvent side view). The positions of the mutations are colored in intermediate gray. The rest of the rRNA is in gray. The ribosomal proteins are omitted for clarity;

FIG. 20 shows the Cluster IX of deleterious mutations in domain II of the 23S rRNA. (A) Secondary structure of the 23S rRNA showing the locations of the mutations. (B) The cluster shown on a backbone model of the D. radiodurans 50S subunit (interface side view). The positions of the mutations are colored in dark gray. The rest of the rRNA is in gray. The ribosomal proteins are omitted for clarity;

FIG. 21 shows the distribution of the deleterious mutations in the (A) small and (B) large ribosomal subunits. Positions of mutations are colored according to the severity of their phenotypes following the scheme of FIG. 1. Proteins are omitted for clarity. For the 30S structure, A-site tRNA, P-site tRNA, and E-site tRNA are shown;

FIG. 22 is a listing of nucleotide sequences used as primers in Examples 1 and 2.

### DETAILED DESCRIPTION OF THE INVENTION

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated.

The present invention discloses a method to map and identify functionally important sites in both the small and the large subunits of the rRNA of the ribosome of a microorganism that represent potential antibiotic targets. The principle of this approach is that antibiotics act upon functional sites, that mutations in the rRNA segments constituting functionally important sites should be deleterious and that such deleterious mutations should therefore identify possible sites of antibiotic action. The experimental strategy for identifying such sites was to generate mutant libraries carrying random mutations in rRNA genes and to select clones whose growth is arrested or diminished when mutant rRNA is expressed and incorporated in the ribosome or the ribosome precursors.

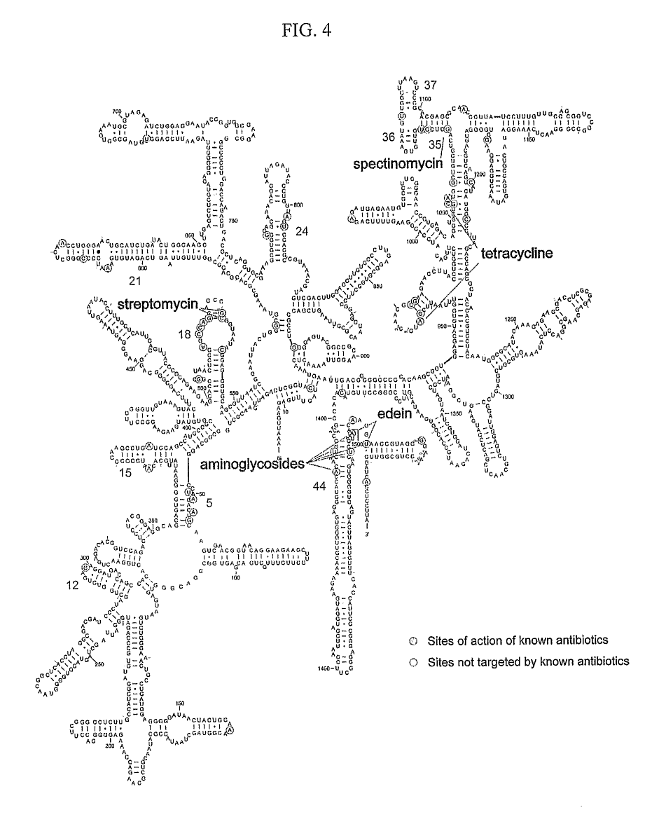

If our main concept, that deleterious rRNA mutations coincide with the potential sites of antibiotic action, is correct, then at least some of the mutations selected from a random library should fall close to the sites of action of known drugs. Indeed, using random mutant libraries of rRNA of the E. coli, we observed clustering of deleterious mutations in rRNA sites associated with the action of such well known antibiotics as aminoglcosides, spectinomycin and tetracycline (FIG. 4) (Carter, A. P., Clemons, W. M., Brodersen, D. E., Morgan-Warren, R. J., Wimberly, B. T. & Ramakrishnan, V. (2000) Nature 407, 340-348; Pioletti, M., Schlunzen, F., Harms, J., Zarivach, R., Gluhmann, M., Avila, H., Bashan, A., Bartels, H., Auerbach, T., Jacobi, C., Hartsch, T., Yonath, A. & Franceschi, F. (2001) EMBO J. 20, 1829-1839; Brodersen, D. E., Carter, A. P., Clemons, W. M., Jr., Morgan-Warren, R. J., Murphy, F. V., Ogle, J. M., Tarry, M. J., Wimberly, B. T. & Ramakrishnan, V. (2001) Cold Spring Harb. Symp. Quant. Biol. 66, 17-32), thus validating our approach. Our second postulate is that the rest of the mapped mutations in the rRNA regions not targeted by the known drugs could be used to identify new antibiotic targets. This postulate is supported by the observation that a number of the mutations that we identified are located in the regions with previously unknown functional importance which are not targeted by any known antibiotics. This notion is further reinforced by our detailed studies of one such rRNA site (position 1111 in 16S rRNA of E. coli) where several deleterious mutations impair functions of rRNA and which, according to our data, may be important for initiation of protein synthesis.

Thus, by mapping deleterious mutations in the rRNA with no known functional significance and are not targeted by any known antibiotics, we have identified new rRNA sites in the bacteria that are critical for efficient translation, and as such could be targeted by antibiotics. Similar approach can also be used to identify new sites of action of protein synthesis inhibitors in the ribosomes of microorganisms other than bacteria as well as in the ribosomes of higher eukaryotes, including humans.

An embodiment of the present invention is a method of mapping and identifying new sites for antibiotic action in the ribosome of a microorganism. The method comprises: (a) providing a random mutant library of the rRNA genes of the microorganism prepared by randomly mutating the rRNA genes of the microorganism; (b) enriching the library in clones with deleterious rRNA mutants by negative selection; (c) screening for clones with deleterious rRNA mutations; (d) mapping the deleterious rRNA mutations in the clones obtained from step (c) to identify sites in the rRNA which are important functional sites in the ribosome; and (e) selecting functional sites identified in the rRNA in step (d) which are not targeted by a known antibiotic as new sites for antibiotic action for the microorganism. The rRNA gene can be that of the rRNA of the small subunit or those of the rRNA of the large subunit of the ribosome of the microorganism.

The steps described above for screening mutants using negative selection are well known to those skilled in the art, except in this case, mutants are screened for deleterious mutations as the phenotype. The general description of these steps can be found in many molecular or microbiology text books such as “Experiments in Molecular Genetics” by Jeffrey H. Miller (Cold Spring Harbor Publisher, 1972) and the steps are illustrated by example of E. coli in the Examples described below.

In an embodiment of the present invention, random mutant libraries are generated by in vivo mutagenesis and fragment exchange in defined segments of a conditionally expressed rRNA gene of the microorganism. Alternatively, random mutant libraries can be generated by other known mutagenesis techniques such as, but are not limited to, the mutagenizing polymerase chain reactions (mutagenizing PCR) used by P. R. Cunningham et al and disclosed in United States Patent Application No.: US2004/0137011 or chemical mutagenesis of the cloned rRNA genes. Such mutagenesis techniques are well known to those skilled in the art. The clones in step (c) of the above method can be identified by replica plating, and deleterious mutations in the ribosome can be mapped and identified by high throughput colony picking, phenotype verification and sequencing.

Although the present invention discloses the mapping and identification of sites for antibiotic action in the ribosome by way of an example in E. coli, it is anticipated that similar approach can be employed to other microorganism species, particularly pathogenic bacteria.

The new sites identified by the method of the present invention can be sites critical for protein synthesis. Mutations in these sites can interfere with protein synthesis either directly by affecting the structure of the function of the ribosome, or indirectly by disrupting the rRNA sites critical for the ribosome assembly. These sites can also be further ranked or classified according to the severity of the deleterious phenotypes, which can be assessed by using the transformation assay. The mutation sites can be ranked or classified into, for example, strongly deleterious, moderately deleterious, and mildly deleterious (see Example 1 below).

Another embodiment of the present invention discloses new functional sites in the rRNA of the ribosome of a microorganism identified by the methods of the present invention. These functional sites represent sites for new antibiotic action in the ribosome of the microorganism.

Yet another embodiment of the present invention discloses a method of screening for new antibiotics by identifying molecules that bind to the new functional ribosomal sites identified by the methods of the present invention to interfere with growth of microorganism. It is important to point out that the binding is between the molecule and the normal, non-mutated sites in the ribosome. The new site is functionally important for the growth of the microorganism. Mutation in this new ribosomal site results in the inhibition of the growth of the microorganism. The binding is not between the molecule and the “mutated” site.

Using the methods described above, we have isolated a large number of deleterious mutations in rRNA of small and large ribosomal subunits. The locations of these mutations highlight new functional centers in the ribosome that can be used as potential drug targets.

As shown in Examples 1 and 2 below, a total of 130 deleterious single-point mutations have been isolated. 53 mutations were mapped in the 16S rRNA and 77 mutations were mapped in the 23S rRNA. The entire collection of rRNA mutations was sorted according to the severity of the deleterious phenotype (see Example 1 for details of the sorting and the classification of the mutations). 28 mutations in 16S and 23S rRNA were associated with a strong deleterious phenotype, 44 mutations caused an intermediate or moderate deleterious phenotype, and 58 mutations were associated with a mild deleterious phenotype. 102 of the 130 mutations are in positions that are 98% or more conserved in the bacterial ribosome, indicating their possible functional relevance. Many of the mutations mapped to regions of well-recognized functional importance located predominantly at the interface side of the small and large ribosomal subunits. A number of mutations were found in sites targeted by known antibiotics, thereby validating our experimental strategy to look for antibiotic sites via mapping sites of deleterious mutations in rRNA.

Ten mutations from the 16S rRNA collection were engineered in the 16S rRNA of the specialized ribosome system, where translation of the reporter mRNA depends entirely on the activity of specialized ribosomes. All of the tested mutations decreased expression of the reporter, indicating that selected mutations affect the ability of the mutant ribosome to participate in protein synthesis.

Deleterious mutations clustered around the known functionally important sites and sites of action of known antibiotics. In addition, a number of deleterious mutations formed clusters in sites of unknown functional significance that are not targeted by the currently known drugs. Four such clusters were identified in the small ribosomal subunit. Clusters I and III were located at the interface side of the subunit whereas clusters II and IV were located at the solvent side. The polysome profile analysis showed that the deleterious effect of mutations in cluster I (mutations A55G and A373G) correlate with a defect in assembly of the small ribosomal subunit. Mutations in cluster IV (the mutations at position A111) conferred a strong functional defect, possibly reflecting impaired initiation of translation by the mutant subunits.

The distribution of the deleterious mutations in the 50S subunit showed a correlation between the severity of the phenotype and the position of the mutations in the subunit structure. A clear gradient of strong to moderate to mild mutations from the interface to the outer surface of the subunit was observed. Four clusters of moderate or mild deleterious mutations were identified in the large ribosomal subunit in the regions of unrecognized functional importance. In addition, two strongly deleterious mutations located in a possibly functionally crucial site in helix 38 of the 23S rRNA not targeted by currently known antibiotics were identified, revealing this site in the large ribosomal subunit as a promising antibiotic target.

The information obtained from preparing and analyzing the collection of deleterious mutations is expected to provide new insights into the ribosome function and reveals new potential sites for antibiotic action in the ribosome. The collection of the mutant clones and the segment-mutant libraries prepared in the course of this work may find applications in studies of the ribosome structure, function, and mechanisms of antibiotic action and drug resistance.

The new functional sites identified by the method of the present invention for antibiotic action in the ribosome can be used to screen or design new antibiotics for microorganisms, particularly pathogenic microorganisms, and more particularly pathogenic bacteria. New antibiotics screening or designing can be accomplished by screening or designing for therapeutic agents that bind to the new ribosomal sites identified by the methods disclosed herein to interfere with the functionality of the sites. Examples of methods for identifying new therapeutic agents binding to a target region in the large ribosomal subunit have been disclosed in U.S. Pat. Nos. 6,947,844, 6,947,845 and 6,952,650 to T. A. Steitz et al. Z. Ma et al. disclose in United States Patent Application No. US2005/0118624 a method for identifying compounds that bind to specific ribosome sites that are known targets of antibiotic action.

In a further embodiment, the present invention discloses a ribosomal site for new antibiotic action for a microorganism. A mutation in the site results in interfering with growth of the microorganism, and the site is not a known target of a known antibiotic.

In yet a further embodiment, the present invention discloses a new antibiotic for a microorganism. The new antibiotic binds to a ribosomal site of the microorganism to interfere with the growth of the microorganism. A mutation in the ribosomal site results in interfering with the growth of the microorganism and the site is not a known target of a known antibiotic.

The articles “a” and “an” are used herein to refer to one or to more than one (i.e., to at least one) of the grammatical object of the article. By way of example, “a molecule” means one molecule or more than one molecule.

“Microorganism” in the present invention includes any microscopic life form which includes but is not limited to bacteria, protozoa, yeasts, fungi, mycobacteria, or mycrobacteria and the like. In a preferred embodiment, the microorganism is a bacterium. In yet another preferred embodiment, the microorganism is pathogenic to a mammalian subject. The mammalian subject is preferably a human subject.

“Antibiotic” as used herein is a therapeutic agent that kills or slows the growth of a microorganism. The therapeutic agent can be a small chemical or a macromolecule. “Macromolecule” includes molecules such as but are not limited to proteins (including polyclonal or monoclonal antibodies), glycoproteins, peptides, carbohydrates (e.g., polysaccharides) or oligonucleotides. The therapeutic agent can be naturally occurring or synthetic. The oligonucleotide can be a DNA or an RNA, including but not limited to antisense oligonucleotides.

The term “mutation” as used herein includes an alteration in the nucleotide sequence of a given gene or regulatory sequence from the naturally occurring or normal nucleotide sequence. A mutation may be a single nucleotide alteration (e.g., deletion, insertion, substitution, including a point mutation), or a deletion, insertion, or substitution of a number of nucleotides.

What is meant by “deleterious mutation” in the present invention is a mutation leading to the interference of cell growth, such as but is not limited to, by inhibiting protein synthesis. The deleterious mutation can be directly interfering with ribosome function, or it can be affecting assembly of the ribosomal subunits.

### EXAMPLES

### Example 1

### Deleterious Mutations in Small Subunit Ribosomal RNA Identify Functional Sites and Potential Targets for Antibiotics

### Materials and Methods

### Enzymes and Chemicals

All of the antibiotics were from Sigma, enzymes were from Fermentas or New England Biolabs, and chemicals were from Fisher Scientific.

Generation of Segment-Mutant rRNA Libraries

The Escherichia coli mutator strain XL-1 Red (Stratagene) was cotransformed with the Kan^{r} plasmid pLG857 (Remaut, E., Stanssens, P. & Fiers, W. (1981) Gene 15, 81-93), carrying a temperature-sensitive λ repressor gene (cI857) and Amp^{r} plasmid pLK45 that carries the E. coli rrnB operon under the control of the λ P_{L} promoter (Powers, T. & Noller, H. F. (1990) Proc. Natl. Acad. Sci. USA 87, 1042-1046). Transformants were selected at 30° C. on LB agar plates containing 100 μg/mL ampicillin and 50 μg/mL kanamycin. Several hundred colonies were washed from the plates. Cells were propagated for 24 hours at 30° C. in 100 mL of LB broth supplemented with antibiotics, and randomly mutated plasmid was prepared.

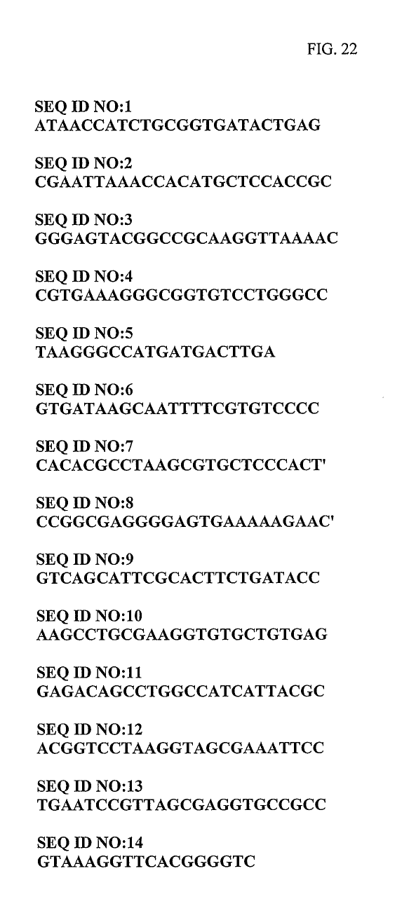

The unique restriction sites of pLK45, KpnI, ApaI, and XbaI were used for a fragment-exchange to generate segment-mutant pLK45 libraries where only specific segments of the plasmid would carry random mutations. The rrnB segment flanked by KpnI and ApaI restriction sites, encompassing the 5′ transcribed spacer and the 930-nucleotide-long 5′ region of the 16S rRNA gene, was PCR amplified using a low-mutation-frequency Triple Master PCR system (Eppendorf) and a pair of primers, ATAACCATCTGCGGTGATACTGAG (SEQ ID NO: 1, FIG. 22) and CGAATTAAACCACATGCTCCACCGC (SEQ ID NO:2, FIG. 22). The amplified PCR fragment was treated with DpnI to remove the template, cut with KpnI and ApaI, and cloned into wild type (wt) pLK45 cut with the same restriction enzymes and treated with calf intestine phosphatase. The resulting segment-mutant library A was transformed into highly competent POP2136 cells, which carry a chromosomal copy of cI857 repressor (Rottmann, N., Kleuvers, B., Atmadja, J. & Wagner, R. (1988) Eur. J. Biochem. 177, 81-90). Transformed cells were grown overnight in ampicillin-LB at 30° C. without prior plating. The analogous procedure was used to produce a segment-mutant library B that carried a randomly mutagenized ApaI-XbaI segment that encompassed a 611-nucleotide-long 3′ segment of the 16S rRNA gene and 182 nucleotides of the 16S/23S spacer. Primers used for PCR amplification of this segment were GGGAGTACGGCCGCAAGGTTAAAAC (SEQ ID NO:3, FIG. 22) and CGTGAAAGGGCGGTGTCCTGGGCC (SEQ ID NO:4, FIG. 22). Segment-mutant libraries were enriched in clones carrying deleterious mutations using negative selection, essentially as described earlier (Tenson, T., Herrera, J. V., Kloss, P., Guameros, G. & Mankin, A. S. (1999) J. Bacteriol. 181, 1617-22), and total plasmid was prepared according to A. Yassin et al. (Yassin A., Fredrick K., and Mankin A. S. (2005) Proc. Natl. Acad. Sci. USA, 102, 16620-16625, Supporting Information).

Screening Segment-Mutant Libraries for Clones with Deleterious rRNA Mutations

The segment-mutant libraries A or B, enriched in clones with deleterious rRNA mutations, were transformed into fresh POP2136 cells and plated on LB/Amp/agar plates. Plates were incubated overnight at 30° C. 8,000 to 12,000 colonies were picked from the plates using a robotic colony picker, and inoculated individually into 90 μL of LB/Amp medium in 384-well plates. After growth at 30° C. for 48 hours each plate was replicated using a 384-pin replicator (Boekel) into 2 new plates, one with LB/Amp medium and the other with LB/Amp medium supplemented with 15 μg/mL erythromycin. Plates were incubated overnight at 30° C. (LB/Amp plate) or at 42° C. (LB/Amp/Ery plate). The A₆₀₀ of the cultures in the plate wells was read using a SPECTRA Max PLUS384 plate reader (Molecular Devices).

Plasmids were prepared from clones that exhibited poor growth at 42° C., and mutant rDNA segments were sequenced from the same pairs of primers that were used for the construction of the corresponding libraries.

The severity of phenotypes conferred by rRNA mutations was tested by transforming plasmids into JM109 cells (Yanisch-Perron, C., Vieira, J. & Messing, J. (1985) Gene 33, 103-119) which were plated at 37° C. or into POP2136 cells plated at 30° C. or 42° C.

### Testing Mutants in the Specialized Ribosome System

Individual 16S rRNA mutations were introduced by fragment exchange using restriction enzymes KpnI and CpoI into the pKF207 plasmid, which contains under the control of an arabinose-inducible promoter the 16S rRNA gene with a mutated anti-Shine-Dalgarno sequence 5′-GGGGU-3′ (Lee, K., Holland-Staley, C. A. & Cunningham, P. R. (1996) RATA 2, 1270-1285; Abdi, N. N. & Fredrick, K. (2005) RNA 11, in press). KLF10 cells [F-ara Δ(gpt-lac) 5λ(ΦPₐₙₜ-SD_{Auccc}-lacz) Kan^{R} srlR301::Tn10 Δ(recA⁻srl)306] that carry a chromosomally encoded lacZ reporter gene with an altered Shine-Dalgamo sequence 5′-AUCCC-3′ were transformed with the resulting plasmids and were plated onto LB/agar plates supplemented with 100 μg of ampicillin. The β-galactosidase activity was determined using the conventional procedure (Miller, J. H. (1992) A Short Course in Bacterial Genetics. Laboratory Manual. (CSHL Press, Cold Spring Harbor)), with some modifications according to A. Yassin et al. (Yassin A., Fredrick K., and Mankin A. S. (2005) Proc. Natl. Acad. Sci. USA, 102, 16620-16625, Supporting Information).

### Polysome Analysis

Polysomes were analyzed following published protocols (Ron, E. Z., Kohler, R. E. & Davis, B. D. (1966) Science 153, 1119-1120), with minor modifications described by A. Yassin et al. (Yassin A., Fredrick K., and Mankin A. S. (2005) Proc. Natl. Acad. Sci. USA, 102, 16620-16625, Supporting Information). The ratio of mutant to wt 16S rRNA in the gradient fractions was determined by primer extension as described in (Sigmund, C. D., Ettayebi, M., Borden, A. & Morgan, E. A. (1988) Methods Enzymol. 164, 673-690), using a primer, AAGGGCCATGATGACTTGA (SEQ NO ID:5, FIG. 22), specific for the pLK45 resident mutation C1192U. Primer-extension products were separated on 12% denaturing polyacrylamide gel and quantified using a Phosphorimager (Molecular Dynamics).

### Results

Selection of Deleterious Mutations in rRNA

Our strategy for identification of functionally and structurally critical sites in the rRNA of the small ribosomal subunit that could be used as antibiotic targets was based on mapping deleterious mutations in 16S rRNA. Conditionally expressed rRNA genes were randomly mutagenized, mutant libraries were enriched in clones with deleterious rRNA mutations, such clones were identified by replica plating, and mutations were mapped by sequencing.

Mutations were generated in the E. coli rrnB operon in the pLK45 plasmid, where it is expressed under the control of the λ P_{L} promoter (Powers, T. & Noller, H. F. (1990) Proc. Natl. Acad. Sci. USA 87, 1042-1046). The plasmid was propagated in E. coli strain POP2136, which carries a chromosomal copy of the temperature-sensitive λ repressor gene. At 30° C., the expression of mutant mRNA genes is abolished; at 42° C. the repressor is inactivated and expression of the plasmid-borne rrnB is induced. The rrnB operon in pLK45 carries a spectinomycin resistance mutation, C1192T, in the 16S rRNA gene and an erythromycin resistance mutation, A2058G, in the 23S rRNA gene that permit monitoring the amount of plasmid-encoded rRNA in the cell. After 3 to 4 hours of induction, plasmid-encoded 16S rRNA accounts for 40% to 60% of the total cellular 16S rRNA (Powers, T. & Noller, H. F. (1990) Proc. Natl. Acad. Sci. USA 87, 1042-1046 and our data, not shown).

Random mutations were introduced into the pLK45 plasmid by propagating it in the E. coli mutator strain XL-1 Red. To avoid counter-selection of deleterious rRNA mutations, pLK45 was cotransformed into the mutator cells together with plasmid pLG857 that encodes temperature-sensitive λ repressor (Remaut, E., Stanssens, P. & Fiers, W. (1981) Gene 15, 81-93), and cells were grown at 30° C. to prevent expression of mutant rRNA. Under the exploited mutagenesis conditions, the expected frequency of mutations is 1 per 2 thousand base pairs (Greener, A., Callahan, M. & Jerpseth, B, (1997) Mol. Biotechnol. 7, 189-195). The initial plasmid library prepared from XL-1 red mutant cells contained approximately 1012 mutant plasmid molecules. Sequencing of the rRNA operon in plasmids prepared from several random clones confirmed the expected frequent presence of multiple mutations. Therefore, to reduce the number of mutations per clone and to facilitate subsequent mutation mapping, secondary (“segment-mutant”) libraries were generated where only a specific portion of the rRNA operon would carry mutations. A 1103-bp-long KpnI-ApaI segment of mutant pLK45 containing the 5′ external transcribed spacer of rrnB and 930 nucleotides of the 5′ portion of the 16S rRNA gene (library A) or a 793-bp-long ApaI-XbaI fragment containing the remaining 3′ segment of the 16S rRNA gene and a part of the 16S-23S intergenic spacer (library B) was introduced into otherwise wt pLK45 by restriction fragment exchange. Each of the segment-mutant libraries contained ca. 5×10⁴ clones. To increase representation of deleterious mutations, the libraries were subjected to 1 round of negative selection, which raised the frequency of clones with deleterious mutations to approximately 8%.

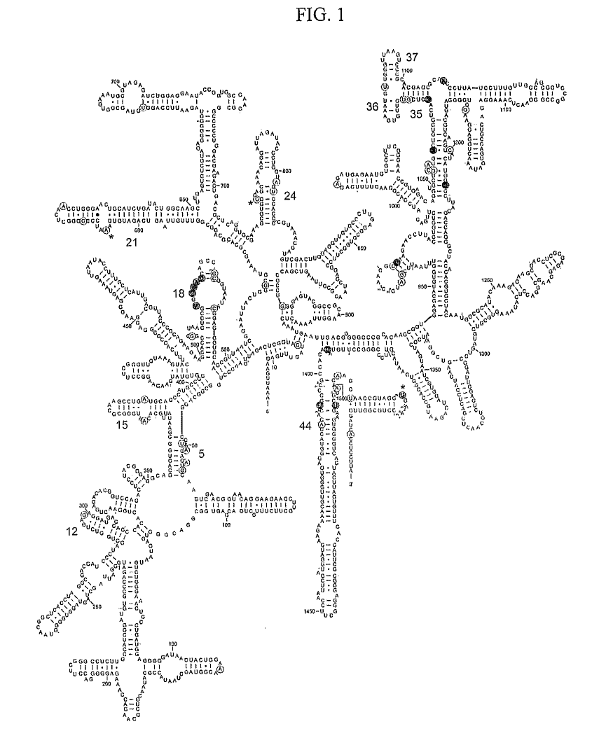

Twelve thousand individual clones of segment-mutant library A and 8,000 clones of library B were individually tested in 384-well plates for their ability to grow at 30° C. (noninduced conditions) or 42° C. (induced). Approximately 400 clones that grew poorly at 42° C. were selected from each library, and after retesting the phenotypes, the mutated segments of the plasmid-borne rrnB were sequenced in a total of 200 clones (FIG. 1 and TABLE 1). The majority of the sequenced clones contained individual point mutations. Some of the mutations were repeatedly found in several independent clones whereas others were represented only in 1 sequenced clone. Clones that contained more than 1 mutation were excluded from further analysis.

TABLE 1
DELETERIOUS MUTATIONS IN E. COLI 16S RIBOSOMAL
RNA SELECTED FROM A RANDOM MUTANT LIBRARY

| | | | | NO. OF |
|---|---|---|---|---|
| | | | | INDEPEN- |
| | | | | DENT |
| MUTATION | DOMAIN | HELIX | PHENOTYPE | CLONES |
| C18G | 5′ | 2 | MODERATE | 1 |
| U49C | 5′ | 5 | MILD | 1 |
| A51G | 5′ | 5 | MILD | 4 |
| A55G | 5′ | 5 | MODERATE | 1 |
| G57A | 5′ | 5 | MILD | 1 |
| A161G | 5′ | 8 | MILD | 1 |
| G299A | 5′ | 12 | MILD | 1 |
| A373G | 5′ | 15 | MILD | 1 |
| A389G | 5′ | 15 | MILD | 2 |
| G506A | 5′ | 18 | MODERATE | 3 |
| Ψ516G | 5′ | 18 | STRONG | 1 |
| C518U | 5′ | 18 | STRONG | 4 |
| C519U | 5′ | 18 | STRONG | 5 |
| A520G | 5′ | 18 | STRONG | 10 |
| G521A | 5′ | 18 | STRONG | 1 |
| G527U | 5′ | 18 | MODERATE | 1 |
| C528U | 5′ | 18 | MODERATE | 2 |
| C536U | 5′ | 18 | MILD | 1 |
| G568C | CENTRAL | 19 | MILD | 1 |
| ΔA (607-609) | CENTRAL | 21 | MILD | 1 |
| C614A | CENTRAL | 21 | MILD | 1 |
| A622G | CENTRAL | 21 | MILD | 1 |
| U684C | CENTRAL | 23 | MILD | 1 |
| ΔG (773-776) | CENTRAL | 24 | MODERATE | 2 |
| A802G | CENTRAL | 24 | MODERATE | 1 |
| U804C | CENTRAL | 24 | MODERATE | 1 |
| G885A | CENTRAL | 27 | MODERATE | 3 |
| G963A | 3′ MAJOR | 31 | MODERATE | 1 |
| A964G | 3′ MAJOR | 31 | MODERATE | 1 |
| C972A/U | 3′ MAJOR | 31 | MODERATE | 2 |
| G973A | 3′ MAJOR | 31 | STRONG | 1 |
| A1014G | 3′ MAJOR | 33 | MILD | 2 |
| G1053A | 3′ MAJOR | 34 | MODERATE | 3 |
| C1054U | 3′ MAJOR | 34 | MILD | 3 |
| A1055G | 3′ MAJOR | 34 | MILD | 1 |
| G1058A | 3′ MAJOR | 34 | STRONG | 7 |
| G1068A | 3′ MAJOR | 35 | STRONG | 2 |
| G1072A | 3′ MAJOR | 35 | MODERATE | 1 |
| U1073C | 3′ MAJOR | 35 | MILD | 1 |
| U1085C | 3′ MAJOR | 37 | MILD | 1 |
| A1111U | 3′ MAJOR | 37 | STRONG | 1 |
| G1181A | 3′ MAJOR | 40 | MODERATE | 1 |
| C1200G | 3′ MAJOR | 34 | MODERATE | 1 |
| C1208G | 3′ MAJOR | 34 | STRONG | 1 |
| C1395U | 3′ MAJOR | 28 | STRONG | 1 |
| U1406C | 3′ MINOR | 44 | STRONG | 3 |
| A1410C | 3′ MINOR | 44 | MODERATE | 1 |
| U1495C | 3′ MINOR | 44 | STRONG | 2 |
| A1499G | 3′ MINOR | 44 | MODERATE | 1 |
| A1502G | 3′ MINOR | 44 | MODERATE | 1 |
| U1506A | 3′ MINOR | 45 | MODERATE | 1 |
| ΔG (1514- | 3′ MINOR | 45 | STRONG | 1 |
| 1517) | | | | |
| A1534G | 3′ MINOR | 45 | MODERATE | 1 |

Deleterious Mutations Identified in 16S rRNA of E. coli

A total of 53 individual point mutations were identified after screening the segment-mutant libraries that span the entire length of the 16S rRNA gene. Even though the adjacent transcribed spacers of rrnB accounted for approximately 20% of the combined mutagenized segments represented in 2 segment-mutant libraries, deleterious mutations were confined exclusively to the mature 16S rRNA sequence (FIG. 1). Of the 53 point mutations, 50 were base substitutions and 3 were single-base deletions.

To rank the mutations, the severity of deleterious phenotypes was assessed using the transformation assay. In POP2136 cells, 16S rRNA transcribed from the pLK45 plasmid at 42° C. accounts for 40% to 60% of the cellular rRNA. Dominant mutations that severely impair protein synthesis prevent colony formation upon transforming the corresponding plasmids into POP2136 cells and incubating the plates at 42° C. E. coli JM109 cells lack λ repressor altogether; as a result, pLK45-encoded 16S rRNA accumulates to up to 85% of the total cellular 16S rRNA. Consequently, even moderately deleterious dominant rRNA mutations are expected to impair formation of JM109 colonies. Thus, according to their ability to transform POP2136 cells (at 42° C.) or JM109 cells (at 37° C.), all of the mutations were grouped into 3 major classes. The first class (black in FIG. 1) included 14 strongly deleterious mutations that failed to transform either POP2136 or JM109 cells. Plasmids carrying any of the 21 moderately deleterious mutations (light gray in FIG. 1) could transform the POP2136 strain but would prevent colony formation in JM109 cells. Finally, 18 mutations of the third class, mildly deleterious (dark gray in FIG. 1), slowed the growth of POP2136 cells in liquid culture at 42° C. but did not prevent colony formation in POP2136 or JM109 cells.

Effect of Selected Small Ribosomal Subunit rRNA Mutations on Protein Synthesis

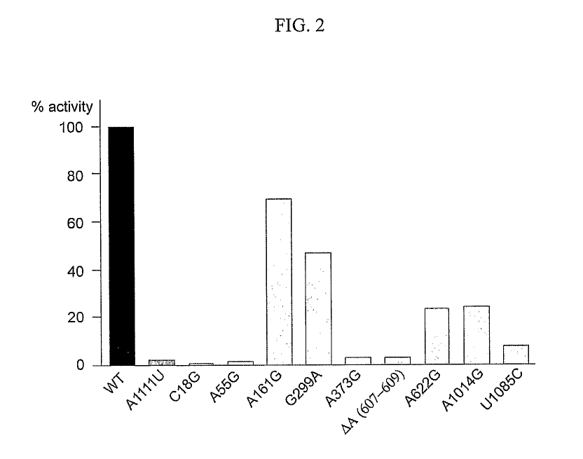

To verify that selected rRNA mutations prevent small ribosomal subunits from participating in protein synthesis, 10 individual mutations were tested in a specialized ribosome system (Lee, K., Holland-Staley, C. A. & Cunningham, P. R. (1996) RNA, 2, 1270-1285; Hui, A. & de Boer, H. A. (1987) Proc. Natl. Acad. Sci. USA 84, 4762-4766). These mutations were engineered in plasmid pKF207, which codes for the 16S rRNA gene with an altered anti-Shine-Dalgarno sequence (GGGGU), and mutant 16S rRNAs were expressed in E. coli strain KLF10, which carries a chromosomal copy of the β-galactosidase gene (lacZ) with a ribosome binding site (AUCCC) recognized by pKF207-encoded 16S rRNA (18). The 30S subunits assembled with the plasmid-encoded 16S rRNA translate only lacZ but not other cellular genes. Therefore, mutations in the 16S rRNA gene in pKF207 do not affect cell growth, whereas the level of β-galactosidase activity reflects the capacity of mutant 16S rRNA to support protein synthesis. In comparison with the control 16S rRNA, which carried only alterations in the Shine-Dalgarno region, all of the mutations engineered in the pKF207 16S rRNA gene reduced reporter expression from approximately 2-fold to 100-fold, confirming that the selected mutations interfered with protein synthesis (FIG. 2). In good correlation with the severity of the phenotype, highly and moderately deleterious mutations (A1111U, C18G, A55G) dramatically reduced expression of the reporter while mildly deleterious mutations had a more variable effect that ranged from intermediate (A161G) to strong (A373G) inhibition of translation.

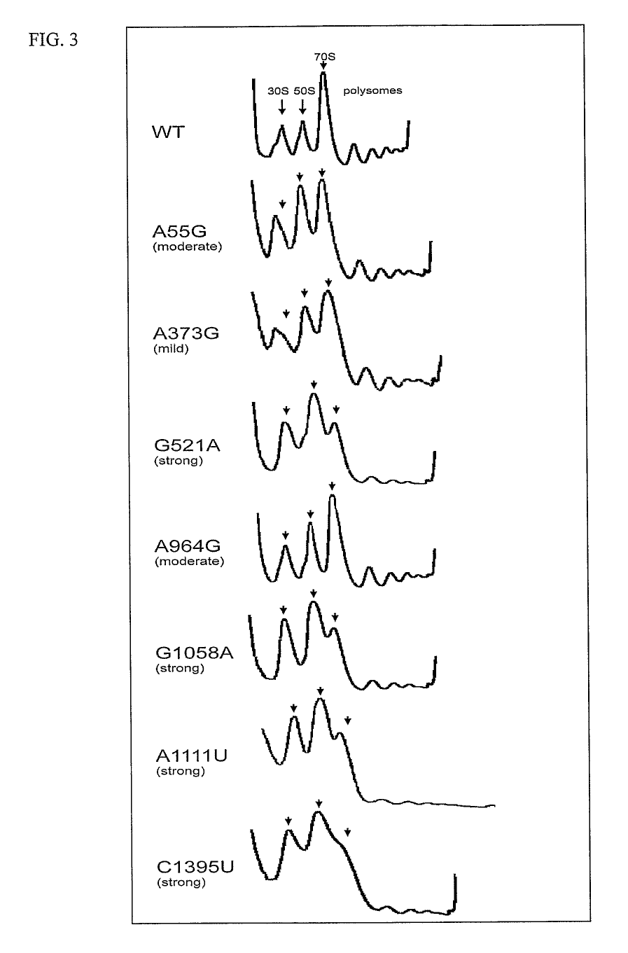

The 16S rRNA mutations can interfere with protein synthesis either directly (by affecting the structure and function of the small ribosomal subunit), or indirectly (by disrupting the rRNA sites critical for the subunit assembly). Both possibilities open up interesting opportunities for development of protein synthesis inhibitors. To understand the general trend of the mode of action of deleterious mutations, we analyzed polysome profiles in several selected POP2136 clones expressing strongly, moderately, or mildly deleterious mutations (FIG. 3). In the only analyzed mildly deleterious mutant, A373G, and in 1 moderately deleterious mutant, A55G, the accumulation of material that sedimented at about 25S and likely represented the aberrant or stalled assembly complexes was clearly seen. This was accompanied by a decreased abundance of 70S ribosomes as compared with free subunits. Primer extension analysis of the A373G mutant showed that mutant 16S rRNA was prevalent in the material sedimenting at 25S (93%), was reduced in the 30S peak (61%), and was notably underrepresented in peaks of 70S ribosomes and polysomes (27% and 22%, respectively), indicating that perturbed ribosome assembly is the primary cause of inhibition of translation and cell growth in these mutants. None of the other 5 strongly or moderately deleterious mutants that were tested (G521A, A964G, G1058A, A1111U, or C1395U) showed any indication of assembly defects. However, all of these mutants showed a marked decrease in the amounts of 70S ribosomes and polysomes relative to free subunits, which is compatible with the idea that the mutations affect functions of the 30S subunits in translation. Thus, it appears that the majority of the identified strongly deleterious and probably moderately deleterious 16S rRNA mutations are associated with functional defects whereas a smaller number of the mutations may interfere with ribosome assembly.

### Discussion

The main goal of this work was to map a variety of functionally important sites in the rRNA of the small ribosomal subunit that represent potential antibiotic targets. By mapping deleterious mutations in E. coli 16S rRNA, we have identified rRNA sites that are critical for efficient translation, and as such could be targeted by antibiotics.

If our main concept, that deleterious rRNA mutations coincide with the potential sites of antibiotic action, is correct, then at least some of the mutations selected from a random library should fall close to the sites of action of known drugs. Indeed, 12 out of 53 of the deleterious mutations identified in 16S rRNA clustered around the sites targeted by well-characterized antibiotics such as aminoglycosides, tetracycline, or streptomycin (FIG. 4) (Carter, A. P., Clemons, W. M., Brodersen, D. E., Morgan-Warren, R. J., Wimberly, B. T. & Ramakrishnan, V. (2000) Nature 407, 340-348; Pioletti, M., Schlunzen, F., Harms, J., Zarivach, R., Gluhmann, M., Avila, H., Bashan, A., Bartels, H., Auerbach, T., Jacobi, C., Hartsch, T., Yonath, A. & Franceschi, F. (2001) EMBO J. 20, 1829-1839; Brodersen, D. E., Carter, A. P., Clemons, W. M., Jr., Morgan-Warren, R. J., Murphy, F. V., Ogle, J. M., Tarry, M. J., Wimberly, B. T. & Ramakrishnan, V. (2001) Cold Spring Harb. Symp. Quant. Biol. 66, 17-32), thus validating our approach. The rest of the mapped mutations were in the rRNA regions not targeted by the known drugs and, thus, could be used to identify new antibiotic targets.

Our experiments were designed to isolate rRNA mutations whose effects would resemble those of an antibiotic bound to the corresponding rRNA sites. When cells are treated with antibiotics, only a certain fraction of ribosomes carries the drug. Potent antibiotics should effectively inhibit protein synthesis, even when a relatively large fraction of ribosomes remains drug-free. The experimental system we used mimicked this situation because in POP2136 cells transformed with the pLK45 plasmid, mutant ribosomes accounted for approximately one half of the ribosomal population. Therefore, the isolated dominant mutations identify ribosomal sites whose distortion diminished cellular translation in spite of the presence of wild type ribosomes. Accordingly, we expect that cells will remain sensitive to the drugs targeted against identified sites even if some of multiple ran alleles in the cell acquire resistance mutations.

Deleterious mutations that were identified in our screening highlight functionally important nucleotides in rRNA. Direct involvement in the function should lead to evolutionary conservation of an rRNA residue. Indeed, the majority of deleterious mutations (48 out of 53) are at the nucleotides that show more than 98% conservation in bacterial 16S rRNA. Therefore, identified nucleotide residues critical in the E. coli ribosome may be functionally important in other bacteria as well and could potentially represent targets for broad-spectrum antibiotics. It should be emphasized, however, that in the absence of experimental data, a mere conservation of a nucleotide is a relatively weak predictor of the extent of its functional importance. More than 600 positions in 16S rRNA exhibit 98% or more conservation (Cannone, J. J., Subramanian, S., Schnare, M. N., Collett, J. R., D'Souza, L. M., Du, Y., Feng, B., Lin, N., Madabusi, L. V., Muller, K. M., Pande, N., Shang, Z., Yu, N. & Gutell, R. R. (2003) BMC.Bioinforinatics 3, 2). A number of mutations engineered at conserved rRNA sites, including those that show conservation across the evolutionary domains, had only weak or even no growth defects (Triman, K. L. (1995) Adv.Genet. 33, 1-39). 42 of the positions that we identified are conserved in the human cytoplasmic ribosomes which raises the question of whether drugs targeted against such sites could be selective. However, as the structures of the ribosome-drug complexes show, antibiotics form multiple contacts with a number of rRNA residues and conservation of one or even several nucleotides is not sufficient to eliminate drug selectivity. As an example, peptidyl transferase targeting antibiotics, many of which show excellent selectivity, act upon the ribosomal site which includes many universally conserved nucleotides.

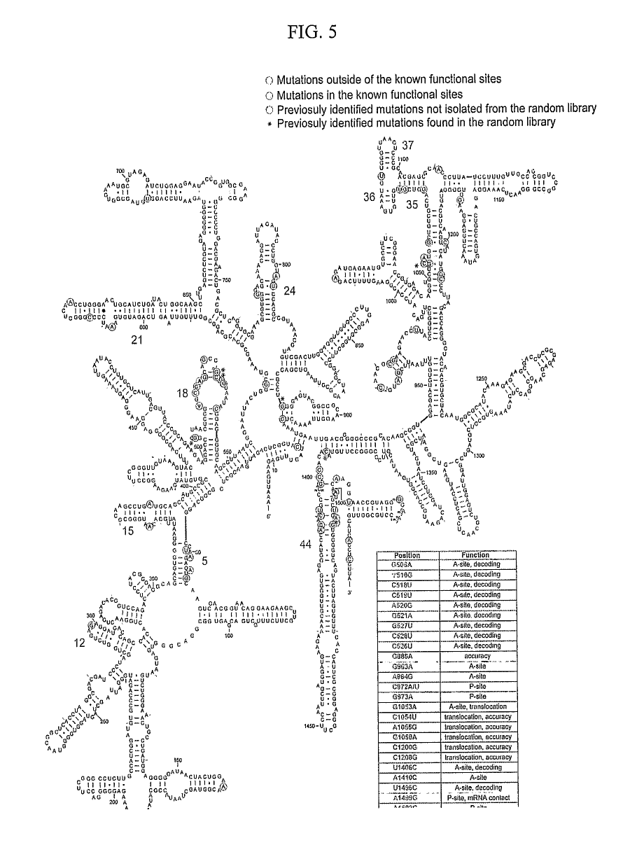

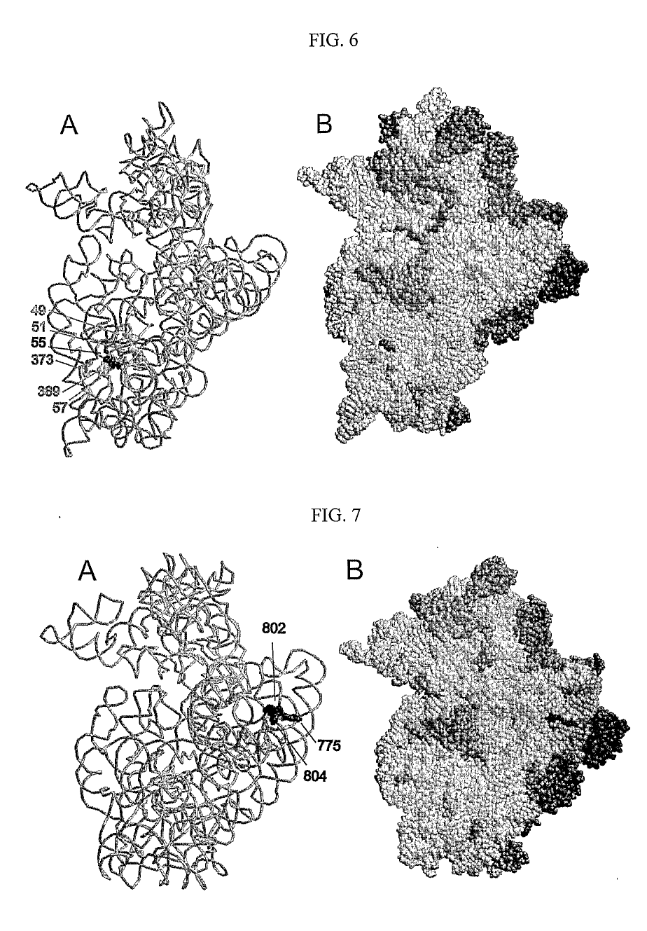

The deleterious mutations were unevenly distributed in the structure of the 30S subunit. While extensive areas of the subunit were virtually mutation-free, several rRNA sites were characterized by clustering of the mutations. Many of the moderate, and even more so, highly deleterious mutations clustered at the functionally charged interface side of the subunit, generally following the path of mRNA and coinciding with the sites of action of several known antibiotics (Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339; Schluenzen, F., Tocilj, A., Zarivach, R., Harms, J., Gluehmann, M., Janell, D., Bashan, A., Bartels, H., Agmon, I., Franceschi, F. & Yonath, A. (2000) Cell 102, 615-623; Yusupova, G. Z., Yusupov, M. M., Cate, J. H. & Noller, H. F. (2001) Cell 106, 233-241). A number of these mutations were localized within known functional sites involved in interactions with tRNA and mRNA, accuracy control, or other ribosome activities (FIG. 5). The possible functional role of the nucleotide positions indicated in the table in FIG. 5 is from the following references: Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339; Schluenzen, F., Tocilj, A., Zarivach, R., Harms, J., Gluehmann, M., Janell, D., Bashan, A., Bartels, H., Agmon, I., Franceschi, F., et al, (2000) Cell 102, 615-623; Noller, H. F., Hoang, L. & Fredrick, K. (2005) FEBS Lett. 579, 855-858; Matassova, A. B., Rodnina, M. V. & Wintermeyer, W. (2001) RNA 7, 1879-1885; Moine, H. & Dahlberg, A. E. (1994) J. Mol. Biol. 243, 402-412; Poldermans, B., Van Buul, C. P. & Van Knippenberg, P. H. (1979) J. Biol. Chem. 254, 9090-9093. Importantly, however, a number of mutations clustered in several regions of less obvious functional significance, regions that can be viewed as putative new antibiotic targets. We identified four such regions. One region, which included mutations in helices 5 (U49C, A51G, A55G, and G57A) and 15 (A373G and A389G), was located at the lower part of the interface side of the subunit (FIG. 6). Though the majority of mutations in this cluster produced only a mild deleterious effect in POP2136 cells (FIG. 1), 2 tested mutations, A55G and A373G, almost completely precluded translation of the reporter protein in the specialized ribosome system (FIG. 2). This seeming controversy is easily reconciled by the observation that A55G and A373G mutations interfere with assembly of 30S subunits. A reduced rate of assembly of the plasmid-encoded 30S subunit may only marginally decrease the total amount of functional ribosomes in the cell when chromosome-encoded wild type ribosomes are present, but it may entirely eliminate translation of the reporter, which entirely relies on the plasmid-encoded 16S rRNA. Our data implicate this otherwise unremarkable region of the ribosome as an important player in ribosome biogenesis and underscores it as one of the putative antibiotic targets in the ribosome.

Three mutations, transitions A802G and U804C and a deletion of 1 out of 4 Gs (773-776), are located in close vicinity of each other in the middle portion of helix 24, which constitutes a part of the intersubunit bridge B7b (Yusupov, M. M., Yusupova, G. Z., Baucom, A., Lieberman, K., Earnest, T. N., Cate, J. H. & Noller, H. F. (2001) Science 292, 883-896) (FIG. 7). This region, which upon subunit association makes a contact with protein L2, was proposed to be part of a signal pathway linking the decoding center of the small ribosomal subunit with the catalytic center of the large subunit (Yusupov, M. M., Yusupova, G. Z., Baucom, A., Lieberman, K., Earnest, T. N., Cate, J. H. & Noller, H. F. (2001) Science 292, 883-896). Clustering of moderately deleterious mutations in this part of helix 24 strongly supports its functional significance. The rRNA mutations, as well as likely binding of small organic molecules at the internal loop of helix 24, may also affect the overall structure of the hairpin, including its apex loop, which may contribute to the binding of tRNA in P and E sites, subunit association, and translation initiation (Tapprich, W. E., Goss, D. J. & Dahlberg, A. E. (1989) Proc. Natl. Acad. Sci. USA 86, 4927-4931; Lee, K. S., Varma, S., SantaLucia, J., Jr. & Cunningham, P. R. (1997) J. Mol. Biol. 269, 732-743).

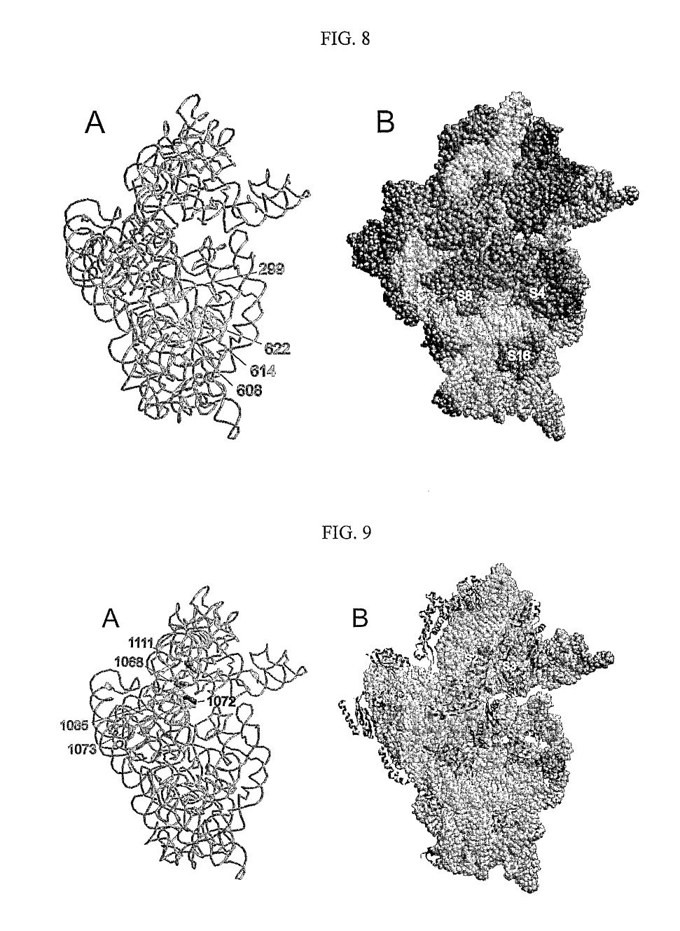

Mutations in helix 21 (C614A, A622G, and deletion of 1 A in a triple-A cluster 607-609) and the G299A mutation in helix 12 converge together at the back of the 30S subunit (FIG. 8). These deleterious mutations are located in close proximity to the ribosomal proteins S4 and S16 whereas the bottom part of helix 21 interacts with ribosomal protein S8. Proteins S4 and S8 are among the primary assembly proteins (Mizushima, S. & Nomura, M. (1970) Nature 226, 1214; Jagannathan, I. & Culver, G. M. (2003) J. Mol. Biol. 330, 373-383; Gregory, R. J. & Zimmermann, R. A. (1986) Nucleic Acids Res. 14, 5761-5776). Thus, similar to the earlier discussed site comprising helices 5 and 15, that might be involved in subunit assembly; the drugs targeted against this site are expected to interfere with formation of functional 30S subunits.

The fourth rRNA site comprises elements of helices 35-37 of the 3′ major domain of 16S rRNA (mutations G1068A, G1072A, U1073C, U1085C, All U). This site is located on the back (solvent) side of the neck of the subunit substantially far from the known functional centers that occupy the interface side. Finding mutations with a strong deleterious effect (G1068A and A1111U) here was unexpected. One of the mutations that was studied in more detail, A 1111U, did not interfere with the subunit assembly but dramatically reduced the fraction of plasmid-encoded 16S rRNA in 70S ribosomes and polysomes (FIG. 3), which indicates severe functional defects associated with this mutation. This conclusion is supported by the inability of mutant 30S subunits to translate the reporter mRNA in the specialized ribosome system (FIG. 2). Accordingly, targeting antibiotics towards this rRNA region is expected to strongly inhibit translation.

How complete is the set of deleterious mutations in 16S rRNA that we have identified? The complexity of the segment-mutant libraries (about 50,000 clones) suggests good coverage of the mutation sequence space. However, a notable bias in favor of transitions vs transversions during in vivo mutagenesis as well as the use of negative selection, which was essential for the success of the project, should inevitably reduce the library complexity. Our collection of point mutations includes 6 of 22 deleterious mutations that were previously engineered or selected in E. coli 16S rRNA using plasmid systems similar to the one used in our experiments (Triman, K. L. & Adams, B. J. (1997) Nucleic Acids Res. 25, 188-191). Thus, we estimate that about 30% of all deleterious mutations in 16S rRNA were obtained in this study. Since deleterious mutations tend to cluster in rRNA sites of high functional significance, we believe that we have found most of the functional sites, despite that our screen was not saturated. Consistent with this, we obtained at least 1 mutation in all the sites specified by the 22 deleterious mutations isolated previously.

The resident mutations present in plasmid pLK45, C1192U in 16S rRNA and A2058G in 23S rRNA, were commonly considered to be silent (Powers, T. & Noller, H. F. (1990) Proc. Natl. Acad. Sci. USA 87, 1042-1046). However, a recent report indicated that they may exhibit synthetic lethality when combined with specific other mutations (Rodriguez-Correa, D. & Dahlberg, A. E. (2004)RNA 10, 28-33). Though we have not tested all the selected mutations in segregation from the resident pLK45 mutations, 10 of the individual deleterious mutations tested in specialized ribosome system showed severe defects in translation, and several other mutations from our collection were previously individually engineered in 16S rRNA and shown to inhibit cell growth (TABLE 1). Therefore, we believe that the majority of the mutations we isolated are genuinely deleterious.

In conclusion, we have isolated an extended collection of deleterious mutations in bacterial 16S rRNA. Clusters of mutations uncovered putative functional regions of the ribosome unrecognized previously. Investigation of these regions may reveal unexpected ribosomal activities and aid in the development of new antibiotics.

### Example 2

### Deleterious Mutations in Large Subunit Ribosomal RNA

### Screening the 23S and 5S Libraries

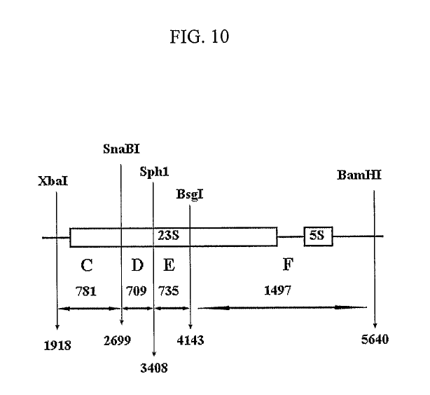

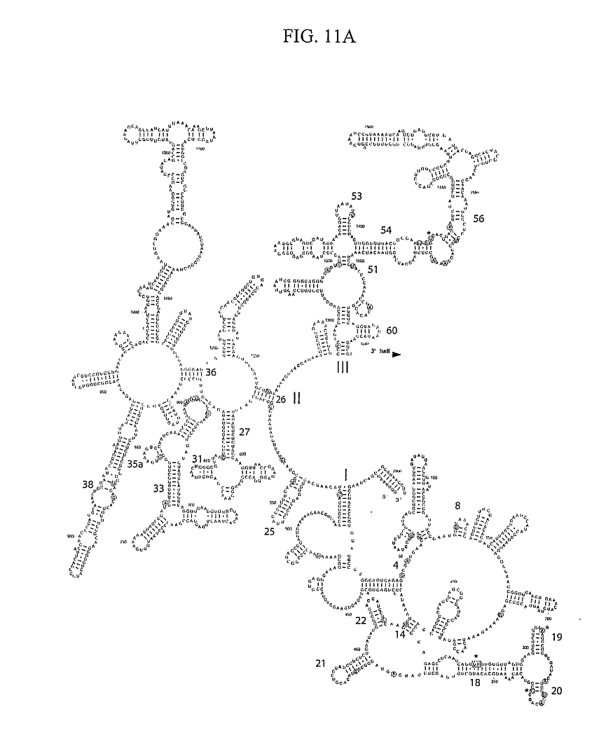

Studies of deleterious mutations in rRNA were continued by selecting mutations in the 23S and 5S rRNA of the large ribosomal subunit. Four segment-libraries covering the entire length of the 23S and 5S rRNA genes were screened for clones expressing a deleterious phenotype (FIG. 10). Library C covered 259 bp of the 16S/23S intergenic spacer and 522 bp of the 23S rRNA gene representing domain I of the rRNA. Library D contained 709 bp of the 23S gene belonging to domain II of the rRNA. Library E included a 735-bp segment of 23S rRNA gene that covered domains III and IV. Finally, library F contained 937 bp of domains V and VI of the 23S rRNA, the 23S/5S spacer, the entire 5S rRNA gene, and the 348-bp spacer following the 5S gene.

Eight thousand clones were screened from library C, and 12,000 clones were screened from each of libraries D, E, and F. The same procedure that was used to screen the 16S libraries was used. The clones were inoculated in 384-well plates using the automated colony picker. After growing for 48 hours at 30° C. in LB/ampicillin, they were tested by replica-plating for their ability to grow at 30° C. (noninduced conditions) or 42° C. (induced). Approximately 400 clones that grew poorly at 42° C. were selected from each library, and after retesting the phenotypes, the mutated segments of the plasmid-borne rrnB were sequenced in a total of 200 clones from each library that had the strongest phenotypes. The majority of the sequenced clones contained individual point mutations. Some of the mutations were repeatedly found in several independent clones whereas others were represented only in one sequenced clone. Only the clones that had single mutations in the rRNA genes were used in subsequent analyses.

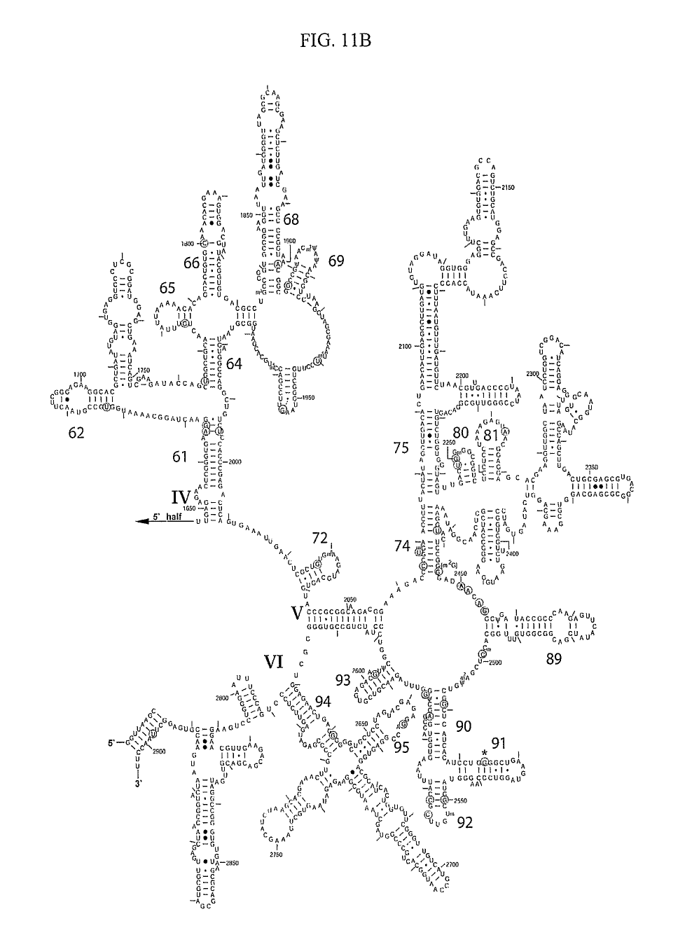

A total of 77 individual point mutations were identified in 23S rRNA. No deleterious mutations were found in the 5S rRNA gene. From the 77 point mutations, 69 were base substitutions and 8 were single-base deletions. Three individual clones carried an additional mutation in the intergenic spacer: one clone from library C (G380A) had a mutation in the 16S/23S spacer (at position 423, numbering from the 3′ end of the 16S), and two clones from library F (A2453G and AC 2556) each had an additional mutation in the 23S/5S spacer (at positions 356 and 321, respectively, numbering from the 3′ end of the 23S). FIGS. 11A and 11B show the locations of the deleterious mutations in the 5′ half and 3′ half of the 23S rRNA, respectively. The helix numbering is according to Yusupov, M. M., et al. (Crystal structure of the ribosome at 5.5 A resolution. Science, 2001. 292: p. 883-896); the secondary structure (Noller, H. F., et al., Secondary structure model for 23S ribosomal RNA. Nucleic Acids Research, 1981. 9: p. 6167-6189) is retrieved from the Web site www.rna.icmb.utexas.edu/(Gutell, R. R., J. C. Lee, and J. J. Cannone, The accuracy of ribosomal RNA comparative structure models. Curr.Opin.Struct.Biol., 2002. 12: p. 301-310).

Ranking of the 23S rRNA Mutations According to the Deleterious Effect

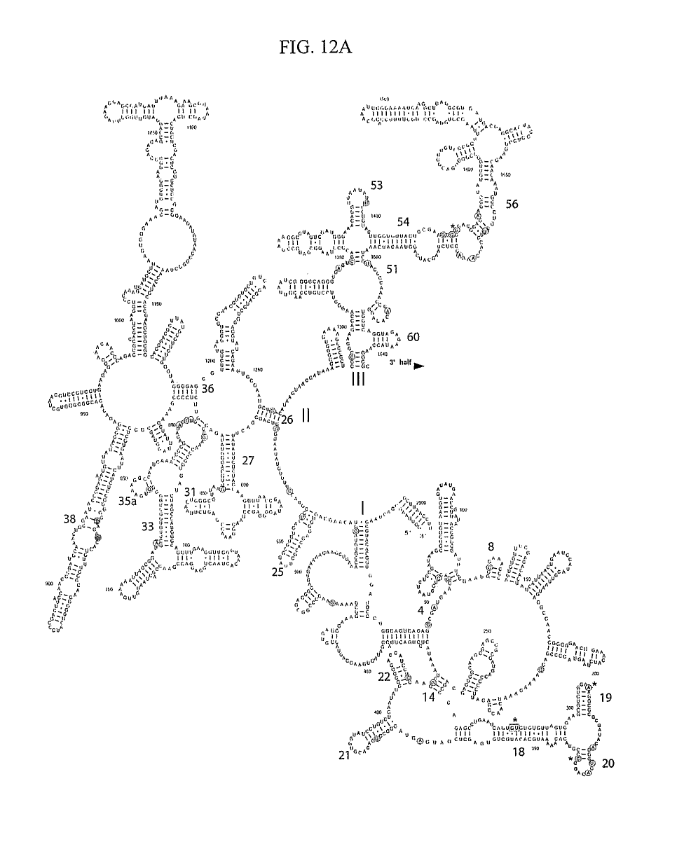

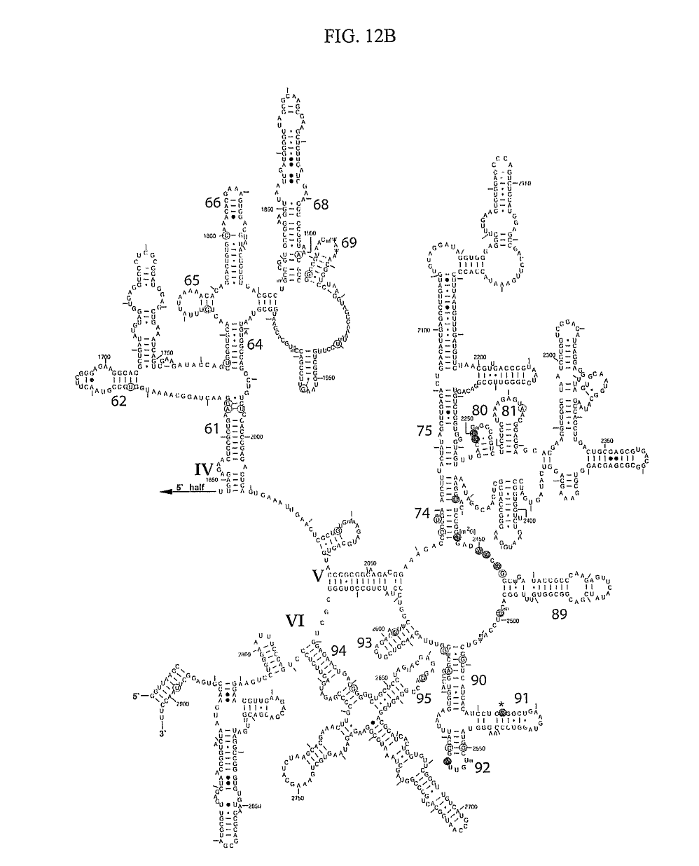

Following the same procedure that was used to sort the 16S rRNA mutations, the mutations in 23S rRNA were ranked into three classes according to their ability to transform POP2136 cells at 42° C. or JM109 cells at 37° C. FIG. 12 (A and B) shows the three classes of deleterious mutations. 14 mutations had strong deleterious phenotypes, as the plasmids carrying the mutations failed to transform both strains (black color). 23 mutations had an intermediate deleterious phenotype; the plasmids carrying these mutations failed to transform E. coli JM109 cells but were able to form colonies in POP2316 cells at 42° C. (light gray color). 40 mutations had a mild deleterious phenotype; POP2136 expressing these mutations grew slowly at 42° C., but both POP2136 and JM109 cells expressing the mutation could form colonies on agar plates (dark gray color). TABLE 2 summarizes the position of each mutation, the type of mutation, the helix and domain where the mutation is present, the severity of the phenotype of each mutation, and the frequency of repeats.

TABLE 2
POSITION, TYPE, LOCATION, SEVERITY OF
PHENOTYPE AND FREQUENCY
OF EACH MUTATION IN THE 23S rRNA

| | | | | | Number of |
|---|---|---|---|---|---|
| | | | | | Independent |
| Position | Clone | Domain | Helix | Phenotype | Clones |
| 46 | G46A | I | 4 | Mild | 1 |
| 49 | A49G | I | 4 | Mild | 1 |
| 55 | G55A | I | 5 | Mild | 1 |
| 58 | G58A | I | 6 | Mild | 1 |
| 123 | G123A | I | 8 | Mild | 1 |
| 215 | G215A | I | 11-12 | Mild | 1 |
| (283-290) | ΔUG (283-290) | I | 18 | Mild | 1 |
| (309-311) | ΔA (309-311) | I | 19 | Mild | 1 |
| 324 | A324G | I | 20 | Mild | 1 |
| 328 | U328C | I | 20 | Mild | 2 |
| 330 | A330G | I | 20 | Mild | 2 |
| (334-337) | ΔC (334-337) | I | 20 | Mild | 8 |
| 371 | A371G | I | 21 | Mild | 1 |
| 380 | G380A | I | 21 | Mild | 1 |
| 420 | C420U | I | 22 | Mild | 1 |
| 425 | G425A | I | 14 | Mild | 2 |
| 481 | G481A | I | 24 | Mild | 1 |
| 523 | ΔC 523 | I | 2 | Mild | 28 |
| 558 | U558A | I | 25 | Mild | 1 |
| 565 | C565U | II | 26 | Moderate | 1 |
| 578 | G578A | II | 26 | Mild | 1 |
| 656 | G656U | II | 27 | Mild | 1 |
| 674 | G674A | II | 32 | Moderate | 6 |
| 764 | A764C | II | 33 | Moderate | 4 |
| 778 | G778A | II | 35a | Mild | 1 |
| 804 | A804G | II | 32-36 | Moderate | 2 |
| 806 | C806U | II | 32-36 | Moderate | 3 |
| 807 | U807C | II | 32-36 | Moderate | 8 |
| 860 | U860C | II | 38 | Strong | 2 |
| 864 | G864A | II | 38 | Strong | 1 |
| 1259 | G1259A | II | 26 | Mild | 1 |
| 1297 | C1297U | III | 48 | Mild | 1 |
| 1342 | A1342G | III | 51 | Mild | 7 |
| 1345 | C1345U | III | 51 | Mild | 1 |
| 1397 | U1397G | III | 53 | Mild | 1 |
| 1421 | G1421A | III | 54 | Mild | 2 |
| 1421/26 | ΔG | III | 54 | Mild | 2 |
| | (G1421-1426) | | | | |
| 1423 | G1423A | III | 54 | Moderate | 1 |
| 1433-34 | ΔA | III | 56 | Mild | 1 |
| | (1433-1434) | | | | |
| 1562 | U1562C | III | 56 | Moderate | 1 |
| 1569 | A1569G | III | 56 | Moderate | 2 |
| 1572 | A1572C | III | 56 | Mild | 1 |
| 1602 | U1602C | III | 51 | Mild | 3 |
| 1614 | A1614G | III | 51 | Mild | 1 |
| 1665 | A1665G | IV | 61 | Moderate | 1 |
| 1666 | G1666A | IV | 61 | Moderate | 1 |
| 1683 | U1683C | IV | 62 | Mild | 1 |
| 1765 | U1765G | IV | 64 | Mild | 1 |
| 1776 | G1776A | IV | 65 | Moderate | 1 |
| 1800 | C1800U | IV | 66 | Mild | 1 |
| 1901 | A1901G | IV | 68 | Moderate | 1 |
| 1907 | G1907A | IV | 69 | Moderate | 2 |
| 1995 | U1995C | IV | 61 | Mild | 1 |
| 2027 | G2027A | V | 72 | Mild | 1 |
| 2065 | C2065U | V | 74 | Moderate | 1 |
| 2068 | U2068G | V | 74 | Moderate | 1 |
| 2249 | U2249A | V | 80 | Strong | 1 |
| 2250 | G2250A | V | 80 | Strong | 1 |
| 2273 | A2273G | V | 81 | Moderate | 1 |
| 2438 | U2438C | V | 74 | Strong | 2 |
| 2446 | G2446A | V | 74 | Strong | 1 |
| 2450 | A2450G | V | 89 | Strong | 2 |
| 2451 | A2451U/G | V | 89 | Strong | 2 |
| 2453 | A2453G | V | 89 | Strong | 1 |
| 2454 | G2454A | V | 89 | Moderate | 1 |
| 2499 | C2499U | V | 89 | Strong | 1 |
| 2509 | G2509A | V | 90 | Moderate | 2 |
| (2523-2526) | ΔG | V | 91 | Strong | 2 |
| | (2523-2526) | | | | |
| 2550 | G2550A | V | 92 | Moderate | 1 |
| 2556 | ΔC 2556 | V | 92 | Strong | 1 |
| 2558 | C2558U | V | 92 | Moderate | 1 |
| 2577 | A2577G | V | 90 | Moderate | 1 |
| 2581 | G2581A | V | 90 | Moderate | 1 |
| 2603 | G2603A | V | 93 | Strong | 1 |
| 2641 | G2641A | VI | 94 | Mild | 1 |
| 2664 | G2664A | VI | 95 | Strong | 1 |
| 2898 | U2898C | VI | 99 | Mild | 1 |

### Phylogenetic Conservation at the Observed Sites

Of 77 23S rRNA positions where deleterious mutations were identified, 54 are more than 98% conserved in bacteria, six are conserved in 90%-98% of bacterial species and two are conserved in 80%-90% of bacteria. Among all of the identified positions in 23S rRNA, 47 are conserved in human cytoplasmic ribosomes (TABLE 3).

TABLE 3
PHYLOGENTIC CONSERVATION OF THE IDENTIFIED
POSITIONS IN THE 23S rRNA TOGETHER WITH
THE HUMAN CELLULAR EQUIVALENT

| | Conservation Among | Human |
|---|---|---|
| Mutation | Bacteria | (Cytoplasmic)* |
| G46A | <80% | C |
| A49G | 98% | U |
| G55A | 98% | A |
| G58A | 98% | G |
| G123A | 98% | A |
| G215A | 98% | G |
| ΔUG (283-290) | <80% | — |
| ΔA (309-311) | 90%-98% | AUC |
| A324G | <80% | A |
| U328C | 98% | U |
| A330G | 98% | U |
| ΔC (334-337) | <80% | GCCG |
| A371G | 98% | A |
| G380A | 80%-90% | G |
| C420U | 80%-90% | C |
| G425A | <80% | G |
| G481A | 98% | G |
| ΔC 523 | 98% | G |
| U558A | <80% | C |
| C565U | 90%-98% | G |
| G578A | <80% | G |
| G656U | 98% | — |
| G674A | 98% | G |
| A764C | 98% | A |
| G778A | 98% | G |
| A804G | 98% | A |
| C806U | 98% | C |
| U807C | 98% | U |
| U860C | 98% | U |
| G864A | 98% | G |
| G1259A | 98% | G |
| C1297U | 90%-98% | C |
| A1342G | 98% | A |
| C1345U | 98% | C |
| U1397G | 98% | C |
| G1421A | <80% | C |
| ΔG (G1421-1426) | <80% | CCGGAG |
| G1423A | <80% | G |
| ΔA (1433-1434) | <80% | GC |
| U1562C | 90%-98% | G |
| A1569G | 98% | G |
| A1572C | 90%-98% | A |
| U1602C | 98% | U |
| A1614G | 98% | C |
| A1665G | 98% | G |
| G1666A | 98% | G |
| U1683C | <80% | A |
| U1765G | <80% | G |
| G1776A | 98% | G |
| C1800U | 98% | A |
| A1901G | 98% | A |
| G1907A | 98% | G |
| U1995C | 98% | C |
| G2027A | 98% | G |
| C2065U | 98% | C |
| U2068G | <80% | U |
| U2249A | 98% | U |
| G2250A | 98% | G |
| A2273G | 98% | A |
| U2438C | 98% | U |
| G2446A | 98% | G |
| A2450G | 98% | A |
| A2451U/G | 98% | A |
| A2453G | 98% | U |
| G2454A | 98% | G |
| C2499U | 98% | U |
| G2509A | 90%-98% | G |
| ΔG (2523-2526) | 98% | UGUG |
| G2550A | 98% | A |
| ΔC 2556 | 98% | C |
| C2558U | 98% | C |
| A2577G | 98% | A |
| G2581A | 98% | G |
| G2603A | 98% | G |
| G2641A | <80% | G |
| G2664A | 98% | G |
| U2898C | 98% | — |

Deleterious Mutations in 23S rRNA

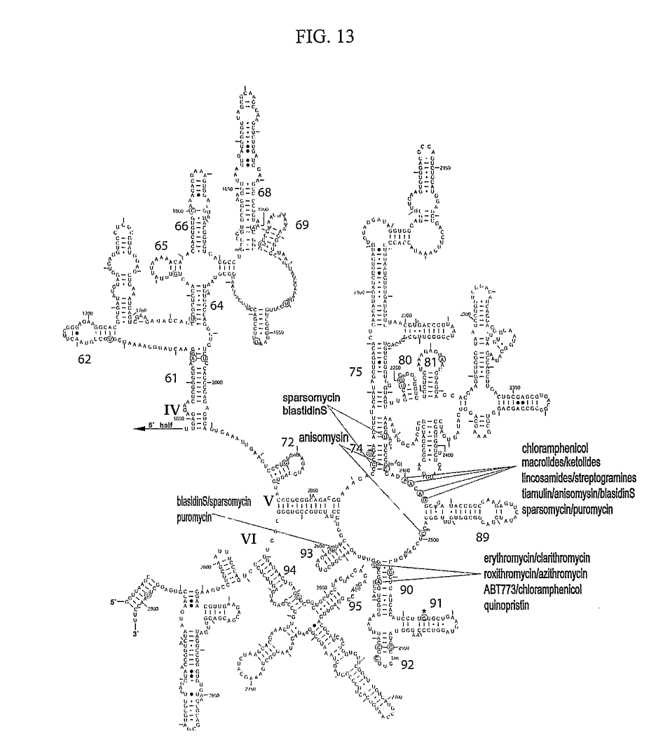

77 deleterious single-point mutations were mapped in 23S rRNA after screening four segment-mutant libraries that cover the entire 23S and 5S rRNA genes. No deleterious mutations were found in the 5S rRNA or in the transcribed spacers. In 23S rRNA, deleterious mutations were found in different domains. However, most of the strongly deleterious mutations were found clustered in and around the central loop of domain V, which constitutes the peptidyl transferase center—the main site of antibiotic action in the large ribosomal subunit. Eleven of the 77 positions are clustered here in the vicinity of the binding sites of macrolides, lincosamides, streptogramines, chloramphenicol, tiamulin, and other antibiotics (FIG. 13) (Schlunzen, F., et al., Structural basis for the interaction of antibiotics with the peptidyl transferase centre in eubacteria. Nature, 2001. 413: p. 814-821; Schlunzen, F., et al., Structural basis for the antibiotic activity of ketolides and azalides. Structure (Camb), 2003. 11(3): p. 329-38; Bashan, A., et al., Structural basis of the ribosomal machinery for peptide bond formation, translocation, and nascent chain progression. Mol Cell, 2003. 11(1): p. 91-102; Berisio, R., et al., Structural Insight into the Antibiotic Action of Telithromycin against Resistant Mutants. J Bacteriol, 2003. 185(14): p. 4276-9; Harms, J. M., et al., Alterations at the peptidyl transferase centre of the ribosome induced by the synergistic action of the streptogram ins dalfopristin and quinupristin. BMC Biol, 2004. 2(1): p. 4; Harms, J. M., et al., Alterations at the peptidyl transferase centre of the ribosome induced by the synergistic action of the streptogramins dalfopristin and quinupristin. BMC Biol, 2004. 2(1): p. 4; Hansen, J., et al., The structures of four macrolide antibiotics bound to the large ribosomal subunit. Mol.Cell, 2002. 10: p. 117-128; Hansen, J. L., P. B. Moore, and T. A. Steitz, Structures of five antibiotics bound at the peptidyl transferase center of the large ribosomal subunit. J Mol Biol, 2003. 330(5): p. 1061-75). As was seen with the collection of 16S rRNA mutations, the rest of the 23S rRNA mutations were mapped to the sites not yet targeted by antibiotics, and consequently, they can potentially identify new antibiotic target sites.

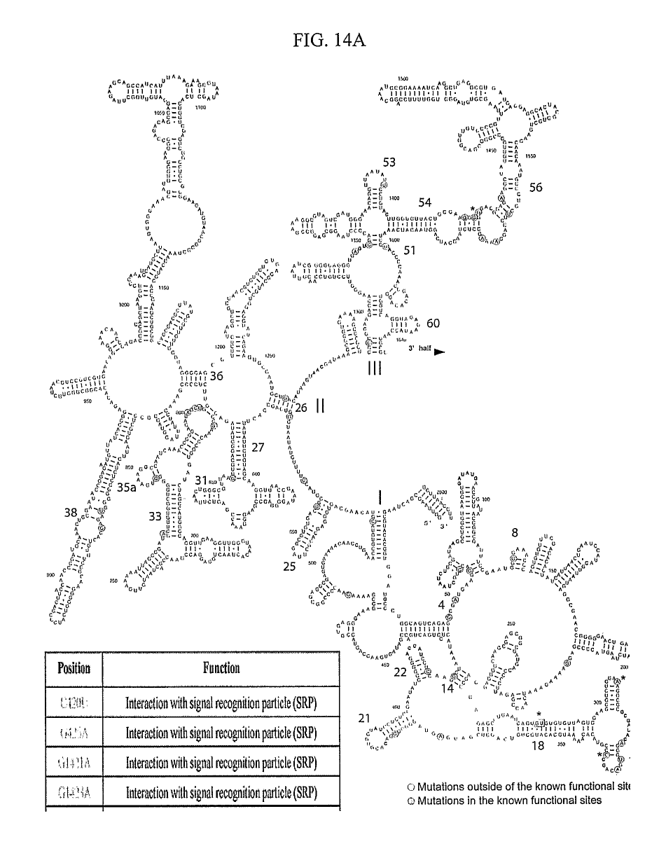

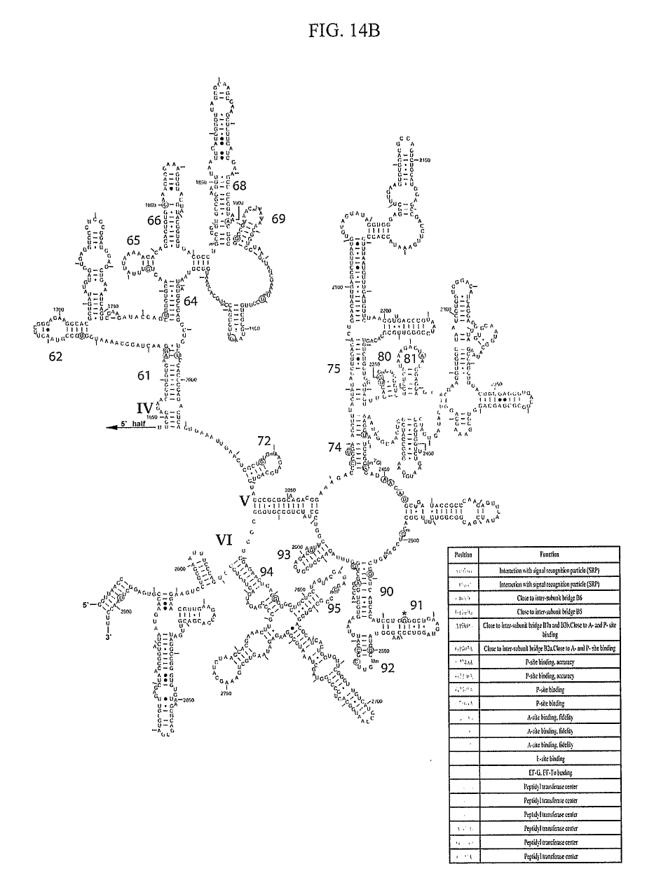

Similar to the collection of mutations in the 16S rRNA, several of the deleterious mutations in the 23S rRNA were located in regions whose functions are understood reasonably well (FIGS. 14A and B) (TABLE 4). Four mutations (U1683C, U1765G, A1901G, and G1907A) belong to the 23S rRNA elements involved in formation of intersubunit bridges (Yusupov, M. M., et al., Crystal structure of the ribosome at 5.5 A resolution. Science, 2001. 292: p. 883-896). Position 1683 is in helix 62, part of the bridge B6. Position 1765 is located in helix 64, which is part of the bridge B5. Position 1901 is between helices 67 and 68, which are part of the intersubunit bridges B2b and B7a, respectively. Position 1907 belongs to helix 69, which is part of the most important bridge (B2a) that is essential for ribosome stability (Maivali, U. and J. Remme, Definition of bases in 23S rRNA essential for ribosomal subunit association. RNA, 2004. 10(4): p. 600-4). Beyond maintaining subunit-subunit associations, at least some of the intersubunit bridges are likely to play important functions in the relative mobility of the subunits and in substrate movement during translocation (Gregory, S. T., et al., Probing ribosome structure and function by mutagenesis. Cold Spring Harb Symp Quant Biol, 2001. 66: p. 101-8; Frank, J. and R. K. Agrawal, A ratchet-like inter-subunit reorganization of the ribosome during translocation. Nature, 2000. 406: p. 318-322). They may also be involved in communicating signals between functional centers of the small and large ribosomal subunits.

TABLE 4
POSITIONS IN THE 23S rRNA WHERE MUTATIONS ARE
IN FUNCTIONAL AREAS AND THE FUNCTION INVOLVED

| Position | Function |
|---|---|
| C420U | Interaction with signal |
| | recognition particle |
| G425A | Interaction with signal |
| | recognition particle |
| G1421A | Interaction with signal |
| | recognition particle |
| G1423A | Interaction with signal |
| | recognition particle |
| A1665G | Interaction with signal |
| | recognition particle |
| G1666A | Interaction with signal |
| | recognition particle |
| U1683C | Intersubunit bridge B6 |
| U1765G | Intersubunit bridge B5 |
| A1901G | Intersubunit bridge B7a and |
| | B2b, close to A- and P-sites |
| G1907A | Intersubunit bridge B2a, close to |
| | A- and P-sites |
| U2249A | P-site, fidelity |
| G2250A | P-site, fidelity |
| G2581A | P-site |
| G2603A | P-site |
| G2550A | A-site, fidelity |
| Δ 2556 | A-site, fidelity |
| C2558U | A-site, fidelity |
| U2438C | E-site, binding |
| G2664A | EF-G, EF-Tu binding |
| G2446A | Peptidyl transferase center |
| A2450G | Peptidyl transferase center |
| A2451U/G | Peptidyl transferase center |
| A2453G | Peptidyl transferase center |
| G2454A | Peptidyl transferase center |
| C2499U | Peptidyl transferase center |

Several other deleterious mutations were localized in the 23S rRNA sites that interact with tRNA (Yusupov, M. M., et al., Crystal structure of the ribosome at 5.5 A resolution, Science, 2001. 292: p. 883-896). Positions 1901 and 1907 are close to the P- and A-site tRNA, which contact the backbones of positions 12 and 13 of the P-site tRNA and of positions 11 and 12 of the A-site tRNA. 23S rRNA positions 2249 and 2250 belong to helix 80, part of the so-called “P-loop,” which establishes Watson-Crick interactions with C74 and C75 of the P-site-bound tRNA (Gregory, S. T., K. R. Lieberman, and A. E. Dahlberg, Mutations in the peptidyl transferase region of E. coli 23S rRNA affecting translation accuracy. Nucleic Acids Research, 1994. 22: p. 279-284; Gregory, S. T. and A. E. Dahlberg, Mutations in the conserved P loop perturb the conformation of two structural elements in the peptidyl transferase center of 23 S ribosomal RNA. Journal of Molecular Biology, 1999. 285: p. 1475-1483). Positions 2581 and 2603 are also close to C75 and A76 of the universally conserved CCA terminus of the P-site-bound tRNA. Positions 2550, 2556, and 2558 are in helix 92, which is part of the A-loop and is critical for the accurate positioning of aminoacyl-tRNA in the peptidyl transferase A-site. G2553 in the A-loop forms a Watson-Crick base pair with C75 of the A-site-bound tRNA. Besides direct effects on tRNA binding, mutations in the P- and A-loops of 23S rRNA may also affect the fidelity of translation (Gregory, S. T. and A. E. Dahlberg, Mutations in the conserved P loop perturb the conformation of two structural elements in the peptidyl transferase center of 23 S ribosomal RNA. Journal of Molecular Biology, 1999. 285: p. 1475-1483; O'Connor, M. and A. E. Dahlberg, Mutations at U2555, a tRNA-protected base in 23S rRNA, affect translational fidelity. Proceedings of the National Academy of Sciences, USA, 1993. 90: p. 9214-9218; Kim, D. F. and R. Green, Base-pairing between 23S rRNA and tRNA in the ribosomal A site. Mol.Cell, 1999. 4: p. 859-864). Positions 2446, 2450, 2451, 2453, 2454, and 2499 are all part of the peptidyl transferase center—the catalytic center of the ribosome responsible for the catalysis of peptide bond formation (Garrett, R. A. and C. Rodriguez-Fonseca, The Peptidyl Transferase Center, in Ribosoinal RATA: Structure, Evolution, Processing, and Function in Protein Biosynthesis, R. A. Zimmermann and A. E. Dahlberg, Editors. 1996, CRC Press: Boca Raton. p. 327-355). Position 2438 belongs to the large subunit E-site and closely approaches the 3′ terminus of the E-site-bound tRNA.

Several 23S rRNA mutations are located in the 50S subunit sites important for the interaction with translation factors and auxiliary proteins. Position 2664 is part of the so-called sarcin-ricin loop, which is critical for the binding of elongation factors EF-G and EF-Tu (Tapprich, W. E. and A. E. Dahlberg, A single base mutation at position 2661 in E. coli 23S ribosomal RNA affects the binding of ternary complex to the ribosome. Embo J, 1990. 9(8): p. 2649-55; Marchant, A. and M. R. Hartley, Mutational studies on the a-sarcin loop of Escherichia coli 23S ribosomal RNA. European Journal of Biochemistry, 1994. 226: p. 141-147; Gluck, A., Y. Endo, and I. G. Wool, The ribosomal RNA identity elements for ricin and for alpha-sarcin: mutations in the putative CG pair that closes a GAGA tetraloop. Nucleic Acids Research, 1994. 22: p. 321-325; Macbeth, M. R. and I. G. Wool, The phenotype of mutations of G2655 in the sarcin/ricin domain of 23 S ribosomal RNA. Journal of Molecular Biology, 1999. 285: p. 965-975; Chan, Y. L., A. S. Sitikov, and I. G. Wool, The phenotype of mutations of the base-pair C2658. G2663 that closes the tetraloop in the sarcin/ricin domain of Escherichia coli 23 S ribosomal RNA. Journal of Molecular Biology, 2000. 298: p. 795-805; Leonov, A. A., et al., Affinity purification of ribosomes with a lethal G2655C mutation in 23S rRNA that affects the translocation. J Biol Chem, 2003). Several mutations (positions 420, 425, 1665, 1668, 1421, and 1423) may affect the interaction of the ribosome with the signal recognition particle (O'Connor, M., et al., Genetic probes of ribosomal RNA function. Biochemistry and Cell Biology, 1995. 73: p. 859-868). This interaction is critical for targeting secreted proteins to the translocon in the bacterial plasma membrane (O'Connor, M., et al., Genetic probes of ribosomal RNA function. Biochemistry and Cell Biology, 1995. 73: p. 859-868; Sagar, M. B., L. Lucast, and J. A. Doudna, Conserved but nonessential interaction of SRP RNA with translation factor EF-G. RNA, 2004. 10(5): p. 772-8; Nagai, K., et al., NEW EMBO MEMBER'S REVIEW. Structure, function and evolution of the signal recognition particle. Embo J, 2003. 22(14): p. 3479-3485).

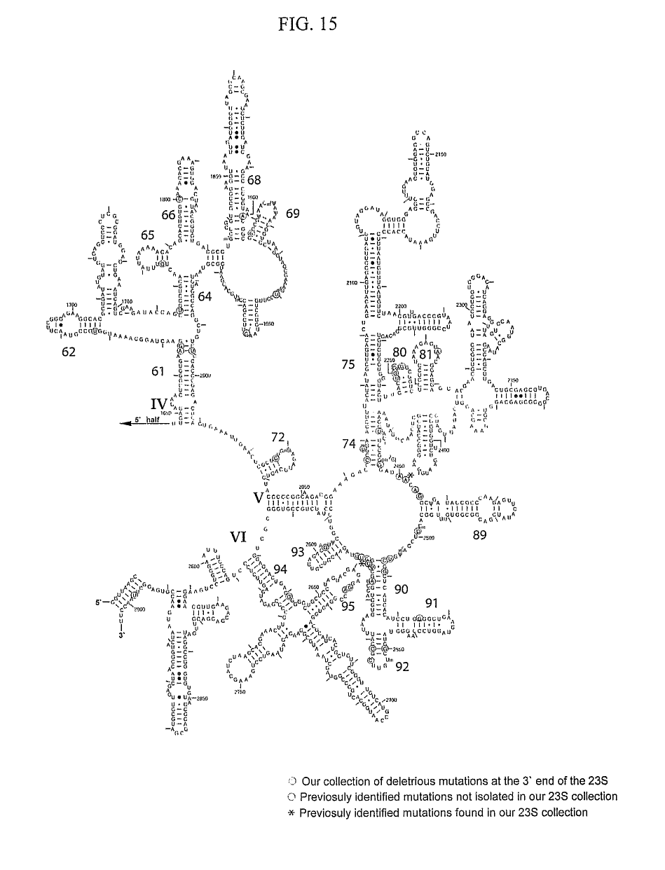

Of the previously identified deleterious mutations in the 23S rRNA studied in experimental systems similar to ours, 11 conferred dominant lethal phenotype (Macbeth, M. R. and I. G. Wool, The phenotype of mutations of G2655 in the sarcin/ricin domain of 23 S ribosomal RNA. Journal of Molecular Biology, 1999. 285: p. 965-975; Green, R., R. R. Samaha, and H. F. Noller, Mutations at nucleotides G2251 and U2585 of 23 S rRNA perturb the peptidyl transferase center of the ribosome. Journal of Molecular Biology, 1997. 266: p. 40-50; Samaha, R. R., R. Green, and H. F. Noller, A base pair between tRNA and 23S rRNA in the peptidyl transferase centre of the ribosome. Nature, 1995. 377: p. 309-314; Spahn, C. M. T., et al., Mutational analysis of two highly conserved UGG sequences of 23 S rRNA from Escherichia coli. Journal of Biological Chemistry, 1996. 271: p. 32849-32856; Porse, B. T., H. P. Thi-Ngoc, and R. A. Garrett, The donor substrate site within the peptidyl transferase loop of 23 S rRNA and its putative interactions with the CCA-end of N-blocked aminoacyl-tRNA^{Phe}. Journal of Molecular Biology, 1996. 264: p. 472-483; Thompson, J., et al., Analysis of mutations at residues A2451 and G2447 of 23S rRNA in the peptidyl transferase active site of the 50S ribosomal subunit. Proceedings of the National Academy of Sciences, USA, 2001. 98: p. 9002-9007; Triman, K. L., A. Peister, and R. A. Goel, Expanded versions of the 16S and 23S Ribosomal RNA Mutation Databases (16SMDBexp and 23SMDBexp). Nucleic Acids Research, 1998. 26: p. 280-284) (FIG. 15). Of these, we found only two in our collection. However, the remaining nine positions mapped very closely to other mutations in our collection, which shows that even if we did not identify every single nucleotide whose mutation confers lethal phenotype, we are likely to identify most of the regions to which dominant lethal mutations map.

Besides mutations located in the known functional centers, a number of mutations were spread throughout the 23S rRNA primary structure. The distribution of these deleterious 23S rRNA mutations in the spatial structure of the large ribosomal subunit was examined using an available crystallographic structure of the eubacterial (Deinococcus radiodurans) 50S subunit (Harms, J., et al., High resolution structure of the large ribosomal subunit from a mesophilic eubacterium. Cell, 2001. 107: p. 679-688). Similar to 16S rRNA, we observed the clustering of some mutations in the large subunit regions whose functional importance is less than obvious. Four such clusters were identified.

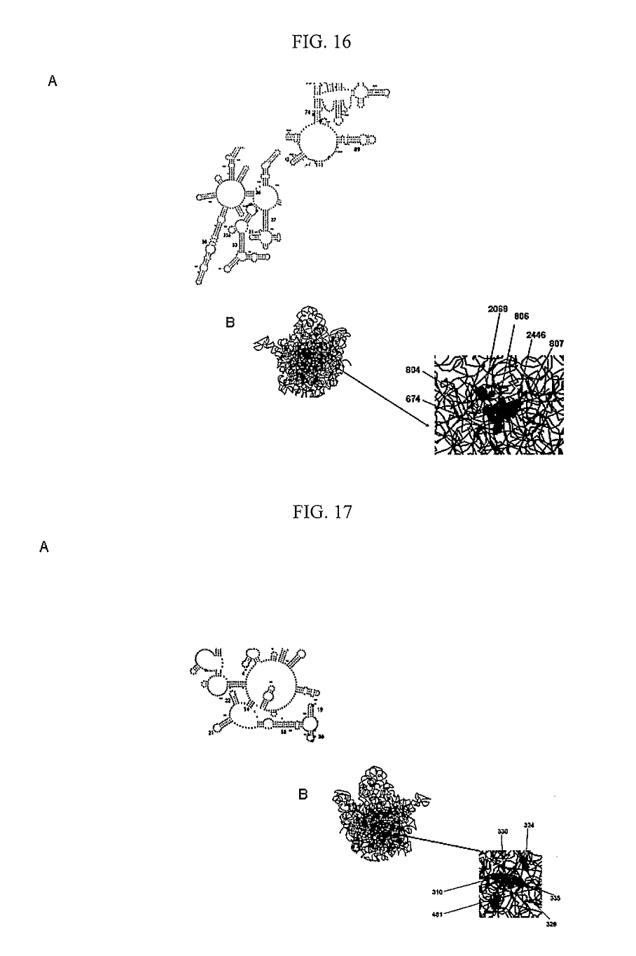

Cluster V (following the numbering adopted for the 16S rRNA) is located on the rim of the peptidyl transferase cavity close to the central protuberance (FIG. 16). It includes mutations at positions 674, 804, 806, and 807 (helix 32) in domain II. These three positions come close to mutations at positions 2068 and 2446 in helix 74 of domain V. All of the positions forming this cluster are highly conserved, even between evolutionary kingdoms, indicating their possible functional relevance. All of these mutations, which exhibited a moderate to strong deleterious phenotype, are located fairly close to the peptidyl transferase active site, and their effect may possibly be explained by allosteric conformational change in the peptidyl transferase center. However, they may also affect tRNA translocation or intersubunit communication.

Cluster VI is located at the back side (solvent side) of the large subunit (FIG. 17). This cluster includes mutations (A324G, U328C, A330G) and deletions (one A from the 309-310 stretch and one C from the 334-337 stretch) in helices 19 and 20 of domain I. Also included in this cluster is the mutation G481A in helix 24. Although the mutations at these positions had mild deleterious phenotypes, their presence at the back (solvent) of the subunit shows the possibility of finding new functional “hot spots” given that most of the known functional regions in the large subunit are located at the interface side. Position 481 is located at a distance ˜13 Å from the orifice of the polypeptide exit tunnel. Positions 324 and 328 are in close proximity to protein L4. Ribosomal proteins L4 and L22 form part of the polypeptide exit tunnel wall, where together they form the narrowest constriction along the tunnel. Some mutations in L4 that lead to an increase in the diameter of the tunnel are associated with macrolide resistance (Gabashvili, I. S., et al., The polypeptide tunnel system in the ribosome and its gating in erythromycin resistance mutants of L4 and L22. EMBO Journal, 2001. 8: p. 181-188; Chittum, H. S, and W. S. Champney, Ribosomal protein gene sequence changes in erythromycin-resistant mutants of Escherichia coli. J Bacteriol, 1994. 176: p. 6192-6198). It was hypothesized that protein L4 may communicate a signal from the ribosome surface to the nascent peptide tunnel. Consequently, mutations at positions 324 and 328 might have an effect on protein L4 and its interaction with the nascent peptide. In addition to its proximity to protein L4, this cluster is reasonably close (5-12 Å) to protein L24, which has a critical role in ribosome assembly (Klein, D. J., P. B. Moore, and T. A. Steitz, The roles of ribosomal proteins in the structure assembly, and evolution of the large ribosomal subunit. J Mol Biol, 2004. 340(1): p. 141-77). Reconstitution experiments have identified protein L24 as one of only two proteins capable of initiation in vitro assembly of the 50S subunit (Nowotny, V. and K. H. Nierhaus, Initiator proteins for the assembly of the 50S subunit from Escherichia coli ribosomes. Proceedings of the National Academy of Sciences, USA, 1982. 79: p. 7238-7242). Mutations in L24 have been associated with slow growth phenotypes and defects of 50S subunit assembly (Dabbs, E. R., A spontaneous mutant of Escherichia coli with protein L24 lacking from the ribosome. Mol Gen Genet, 1982. 187(3): p. 453-8; Cabezon, T., et al., Ribosomal assembly deficiency in an Escherichia coli thermosensitive mutant having an altered L24 ribosomal protein. J Mol Biol, 1977. 116(3): p. 361-74). Mutations in this cluster might therefore affect the functions of protein L24 and, hence, interfere with assembly of the large subunit.

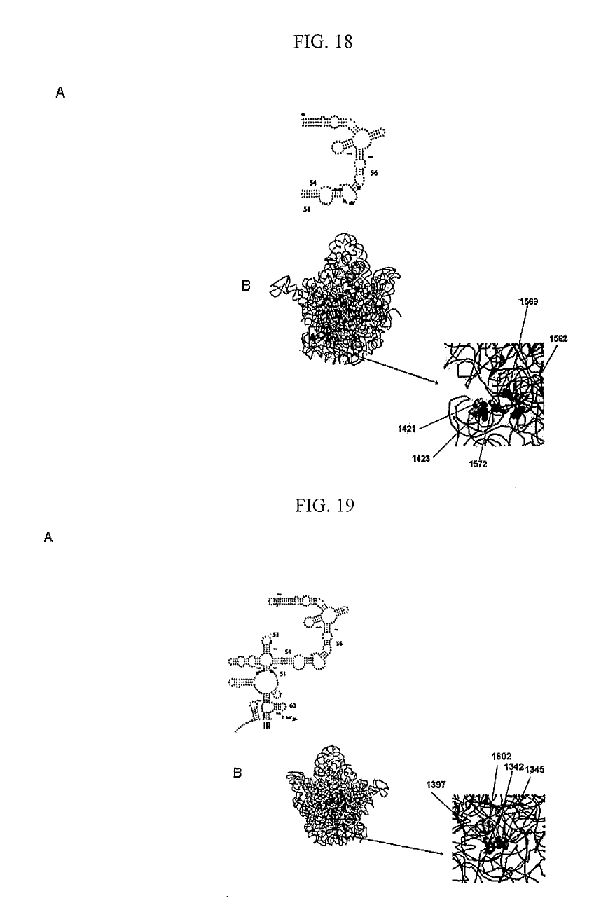

Cluster VII includes mutations in domain III of 23S rRNA (1421 and 1423 in helix 54 and 1562, 1569, and 1572 in helix 56). This cluster is located at the lower left of the interface side of the large subunit (FIG. 18). This cluster is positioned close to the location of protein L2. Protein L2 is one of the early assembly proteins. It binds to 23S rRNA in an early assembly step prior to the formation of any RNA tertiary interactions and drives the subunit assembly (Klein, D. J., P. B. Moore, and T. A. Steitz, The roles of ribosomal proteins in the structure assembly, and evolution of the large ribosomal subunit. J Mol Biol, 2004. 340(1): p. 141-77). Thus, one would not be surprised to find that mutations at this cluster might cause the assembly defect. On the other hand, protein L2 is important for the formation of the peptidyl transferase center and possibly its activity (Diedrich, G., et al., Ribosomal protein L2 is involved in the association of the ribosomal subunits, tRNA binding to A and P sites and peptidyl transfer. EMBO Journal, 2000. 19: p. 5241-5250; Khaitovich, P., et al., Reconstitution of functionally active Thermus aquaticus large ribosomal subunits with in vitro-transcribed rRNA. Biochemistry, 1999. 38: p. 1780-1788). Thus, it would be interesting to test the peptidyl transferase activity of the ribosome carrying mutations in Cluster VII in the future.

Cluster VIII is located at the back (solvent) side of the large subunit. Mutations at positions 1342, 1345, and 1602 of helix 51 approach the site of a mutation at position 1397 in helix 53; they are all part of domain III (FIG. 19). Positions 1345 and 1602 are conserved even in the mitochondrial equivalent of the 23S rRNA, indicating possible functional relevance. All of the mutations belonging to this cluster are in close proximity (5-12 Å) to the ribosomal protein L23. Together, ribosomal proteins L23 and L29 flank the exit of the polypeptide exit tunnel. Protein L23 acts as a chaperone docking site on the large subunit (Kramer, G., et al., L23 protein functions as a chaperone docking site on the ribosome. Nature, 2002. 419(6903): p. 171-4). For example, it interacts with trigger factor, a cytosolic chaperone that assists folding and prevents aggregation of nascent peptides (Jenni, S, and N. Ban, The chemistry of protein synthesis and voyage through the ribosomal tunnel. Curr Opin Struct Biol, 2003. 13(2): p. 212-9). Protein L23a (the eukaryotic equivalent of L23) has been cross-linked to the SRP54 domain of the signal recognition particle (Pool, M. R., et al., Distinct modes of signal recognition particle interaction with the ribosome. Science, 2002. 297(5585): p. 1345-8). Consequently, the mutations at this cluster may possibly have an effect on the conformation of protein L23 and the interaction of the ribosome with the chaperone and secretion machineries.

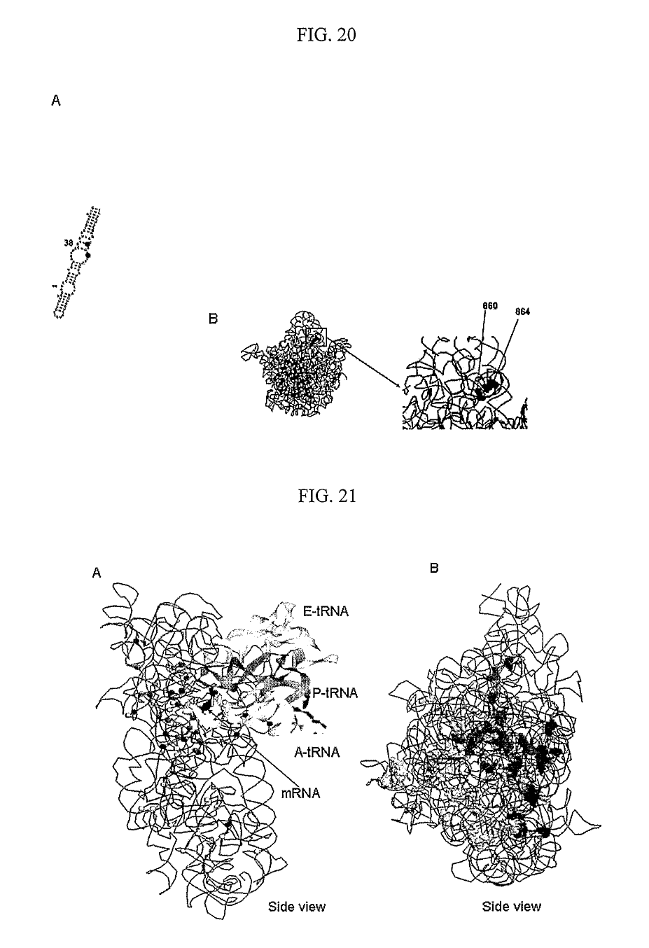

Though we define a “cluster” as a site where three or more mutations come close together in the ribosome three-dimensional structure, we isolated two of the mutations in our collection into an independent cluster (cluster IX). The location of this cluster is defined by two mutations mapped to helix 38 (U860C and G864A) (FIG. 20). These two mutations are among the strongest deleterious mutations in our 23S collection. They belong to a so-called “A-site finger”—one of the intersubunit bridges (B1a) that was hypothesized to be involved in translocation even though its functions remain largely unclear. Interestingly, although deletion of the distal part of helix 38 led to a less efficient subunit association, it had no major effects on tRNA binding, translocation, or cell growth characteristics (Sergiev, P. V., et al., The conserved A-site finger of the 23S rRNA: just one of the intersubunit bridges or a part of the allosteric communication pathway? J Mol Biol, 2005. 353(1): p. 116-23). And yet, our two mutations in the internal loop of helix 38 have a profound dominant negative effect on cell growth. Future studies of this site using the deleterious mutations U860C and G864A as an experimental tool might help explain the role that this rRNA element plays in translation. In any event, owing to the strong phenotype of these mutations and the idiosyncratic structure of the RNA element they belong to, the ribosomal site identified by cluster IX is viewed as a promising new antibiotic site in the large ribosomal subunit.

### Distribution of the Mutations in the Small and Large Subunits

The identified deleterious mutations in 16S and 23S rRNA were rather unevenly distributed in the three-dimensional structure of both subunits. A closer look at the distribution of the mutations reveals a clear common tendency for the mutations with strong or moderate deleterious phenotypes to concentrate in a fairly small area at the interface sides of the subunits, whereas the mutations with mild deleterious phenotypes tend to aggregate close to the subunits' solvent sides (FIGS. 21A and B).

For the small subunit, most of the strong and moderate deleterious mutations were localized around the path of mRNA on the subunit interface side, especially at the sites of codon-anticodon interactions of the three tRNAs (FIG. 21A). The strong and moderate deleterious mutations can be seen as forming a cleft surrounding the mRNA and the anticodon loops of the three tRNAs (FIG. 21A). Moderately deleterious mutations formed the second shell of deleterious mutations whereas mildly deleterious mutations were located at a larger distance from the main functional centers—mostly at the lower portion of the subunit body. Only a few strongly deleterious mutations were localized on the back of the subunit and particularly at the junction between the head and the body. Given the structural changes that the small subunit undergoes during translation initiation, decoding, and translocation that all involve movement of the head, it is conceivable that mutations in this area, including a very interesting cluster IV that we discussed earlier, would prevent functionally critical conformational transitions.

For the large subunit, most of the strong and moderately deleterious mutations were again located at the functionally charged interface of the subunit clustering close to the peptidyl transferase center. The strong deleterious mutations mapped almost exclusively to the A- and P-sites (except for the cluster IX discussed earlier, which is located at the subunit interface above the peptidyl transferase cavity). The severity of the mutations progressively decreases as we move from the interface to the solvent side (FIG. 21B). No strongly deleterious mutations and very few mildly deleterious mutations were found at the solvent side of the large ribosomal subunit. This observation correlates very well with the fact that only a few of the known antibiotics that act on the large ribosomal subunit target sites other than the peptidyl transferase center.

The practice of the present invention will employ and incorporate, unless otherwise indicated, conventional techniques of cell biology, cell culture, molecular biology, microbiology, genetic engineering, and immunology, which are within the skill of the art. While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein.

### REFERENCES

1. Ramakrishnan, V. (2002) Cell 108, 557-572.
2. Cundliffe, E. (1990) in The Ribosome: Structure, Function, & Evolution, eds. Hill, W. E., Dahlberg, A., Garrett, R. A., Moore, P. B., Schlessinger, D. & Warner, J. R. (American Society for Microbiology, Washington, D.C.), pp. 479-490.
3. Sigmund, C. D., Ettayebi, M., Borden, A. & Morgan, E. A. (1988) Methods Enzymol. 164, 673-690.
4. Farrell, D. J., Douthwaite, S., Morrissey, I., Bakker, S., Poehlsgaard, J., Jakobsen, L. & Felmingham, D. (2003) Antimicrob. Agents Chemother. 47, 1777-1783.
5. Wright, G. D. (2003) Curr. Opin. Chem. Biol. 7, 563-569.
6. Cundliffe, E. (1987) Biochimie 69, 863-869.
7. Garrett, R. A. & Rodriguez-Fonseca, C. (1996) in Ribosomal RNA. Structure, Evolution, Processing, and Function in Protein Biosynthesis, eds. Zimmermann, R. A. & Dahlberg, A. E. (CRC Press, Boca Raton), pp. 327-355.
8. Adrian, P. V., Mendrick, C., Loebenberg, D., McNicholas, P., Shaw, K. J., Klugman, K. P., Hare, R. S. & Black, T. A. (2000) Antimicrob. Agents Chemother. 44, 3101-3106.
9. Belova, L., Tenson, T., Xiong, L., McNicholas, P. M. & Mankin, A. S. (2001) Proc. Natl. Acad. Sci. USA 98, 3726-31.
10. Laios, E., Waddington, M., Saraiya, A. A., Baker, K. A., O'Connor, E., Pamarathy, D. & Cunningham, P. R. (2004) Arch. Pathol. Lab. Med. 128, 1351-1359.
11. Triman, K. L. & Adams, B. J. (1997) Nucleic Acids Res. 25, 188-191.
12. Remaut, E., Stanssens, P. & Fiers, W. (1981) Gene 15, 81-93.
13. Powers, T. & Noller, H. F. (1990) Proc. Natl. Acad. Sci. USA 87, 1042-1046.
14. Rottmann, N., Kleuvers, B., Atmadja, J. & Wagner, R. (1988) Eur. J. Biochem. 177, 81-90.
15. Tenson, T., Herrera, J. V., Kloss, P., Guarneros, G. & Mankin, A. S. (1999) J. Bacteriol 181, 1617-22.
16. Yanisch-Perron, C., Vieira, J. & Messing, J. (1985) Gene 33, 103-119.
17. Lee, K., Holland-Staley, C. A. & Cunningham, P. R. (1996) RNA 2, 1270-1285.
18. Abdi, N. N. & Fredrick, K. (2005) RNA 11, in press.
19. Miller, J. H. (1992) A Short Course in Bacterial Genetics. Laboratory Manual. (CSHL Press, Cold Spring Harbor).
20. Ron, E. Z., Kohler, R. E. & Davis, B. D. (1966) Science 153, 1119-1120.
21. Greener, A., Callahan, M. & Jerpseth, B. (1997) Mol. Biotechnol. 7, 189-195.
22. Hui, A. & de Boer, H. A. (1987) Proc. Natl. Acad. Sci. USA 84, 4762-4766.
23. Carter, A. P., Clemons, W. M., Brodersen, D. E., Morgan-Warren, R. J., Wimberly, B. T. & Ramakrishnan, V. (2000) Nature 407, 340-348.
24. Pioletti, M., Schlunzen, F., Harms, J., Zarivach, R., Gluhmann, M., Avila, H., Bashan, A., Bartels, H., Auerbach, T., Jacobi, C., Hartsch, T., Yonath, A. & Franceschi, F. (2001) EMBO J. 20, 1829-1839.
25. Brodersen, D. E., Carter, A. P., Clemons, W. M., Jr., Morgan-Warren, R. J., Murphy, F. V., Ogle, J. M., Tarry, M. J., Wimberly, B. T. & Ramakrishnan, V. (2001) Cold Spring Harb. Symp. Quant. Biol. 66, 17-32.
26. Cannone, J. J., Subramanian, S., Schnare, M. N., Collett, J. R., D'Souza, L. M., Du, Y., Feng, B., Lin, N., Madabusi, L. V., Muller, K. M., Pande, N., Shang, Z., Yu, N. & Gutell, R. R. (2003) BMC.Bioinformatics 3, 2.
27. Triman, K. L. (1995) Adv.Genet. 33, 1-39.
28. Wimberly, B. T., Brodersen, D. E., Clemons, W. M., Jr., Morgan-Warren, R. J., Carter, A. P., Vonrhein, C., Hartsch, T. & Ramakrishnan, V. (2000) Nature 407, 327-339.
29. Schluenzen, F., Tocilj, A., Zarivach, R., Harms, J., Gluehmann, M., Janell, D., Bashan, A., Bartels, H., Agmon, I., Franceschi, F. & Yonath, A. (2000) Cell 102, 615-623.
30. Yusupova, G. Z., Yusupov, M. M., Cate, J. H. & Noller, H. F. (2001) Cell 106, 233-241.
31. Yusupov, M. M., Yusupova, G. Z., Baucom, A., Lieberman, K., Earnest, T. N., Cate, J. H. & Noller, H. F. (2001) Science 292, 883-896.
32. Tapprich, W. E., Goss, D. J. & Dahlberg, A. E. (1989) Proc. Natl. Acad. Sci. USA 86, 4927-4931.
33, Lee, K. S., Varma, S., SantaLucia, J., Jr. & Cunningham, P. R. (1997) J. Mol. Biol. 269, 732-743.
34. Mizushima, S. & Nomura, M. (1970) Nature 226, 1214.
35. Jagannathan, I. & Culver, G. M. (2003) J. Mol. Biol. 330, 373-383.
36. Gregory, R. J. & Zimmermann, R. A. (1986) Nucleic Acids Res. 14, 5761-5776.
37. Rodriguez-Correa, D. & Dahlberg, A. E. (2004) RNA 10, 28-33.
38. Woese, C. R., Magrum, L. J., Gupta, R., Siegel, R. B., Stahl, D. A., Kop, J., Crawford, N., Brosius, J., Gutell, R., Hogan, J. J. & Noller, H. F. (1980) Nucleic Acids Res. 8, 2275-2293.
39. Brodersen, D. E., Clemons, W. M., Jr., Carter, A. P., Morgan-Warren, R. J., Wimberly, B. T. & Ramakrishnan, V. (2000) Cell 103, 1143-1154.
40. Noller, H. F., Hoang, L. & Fredrick, K. (2005) FEBS Lett. 579, 855-858.
41. Matassova, A. B., Rodnina, M. V. & Wintermeyer, W. (2001) RNA 7, 1879-1885.
42. Moine, H. & Dahlberg, A. E. (1994) J. Mol. Biol. 243, 402-412.
43. Poldermans, B., Van Buul, C. P. & Van Knippenberg, P. H. (1979) J. Biol. Chem. 254, 9090-9093.
44. Noller, H. F., et al., Secondary structure model for 23S ribosomal RNA. Nucleic Acids Research, 1981.9: p. 6167-6189.
45. Gutell, R. R., J. C. Lee, and J. J. Cannone, The accuracy of ribosomal RNA comparative structure models. Curr.Opin.Struct.Biol., 2002. 12: p. 301-310.
46. Schlunzen, F., et al., Structural basis for the interaction of antibiotics with the peptidyl transferase centre in eubacteria. Nature, 2001. 413: p. 814-821.
47. Schlunzen, F., et al., Structural basis for the antibiotic activity of ketolides and azalides. Structure (Camb), 2003. 11(3): p. 329-38.
48. Bashan, A., et al., Structural basis of the ribosomal machine for peptide bond formation, translocation, and nascent chain progression. Mol Cell, 2003. 11(1): p. 91-102.
49. Berisio, R., et al., Structural Insight into the Antibiotic Action of Telithromycin against Resistant Mutants. J Bacteriol, 2003. 185(14): p. 4276-9.
50. Harms, J. M., et al., Alterations at the peptidyl transferase centre of the ribosome induced by the synergistic action of the streptogramins dalfopristin and quinupristin. BMC Biol, 2004. 2(1): p. 4.
51. Harms, J. M., et al., Alterations at the peptidyl transferase centre of the ribosome induced by the synergistic action of the streptogramins dalfopristin and quinupristin. BMC Biol, 2004. 2(1): p. 4.
52. Hansen, J., et al., The structures of four macrolide antibiotics bound to the large ribosomal subunit. Mol.Cell, 2002. 10: p. 117-128.
53. Hansen, J. L., P. B. Moore, and T. A. Steitz, Structures of five antibiotics bound at the peptidyl transferase center of the large ribosomal subunit. J Mol Biol, 2003. 330(5): p. 1061-75.
54. Maivali, U. and J. Remme, Definition of bases in 23S rRNA essential for ribosomal subunit association. RNA, 2004. 10(4): p. 600-4.
55. Gregory, S. T., et al., Probing ribosome structure and function by mutagenesis. Cold Spring Harb Symp Quant Biol, 2001. 66: p. 101-8.
56. Frank, J. and R. K. Agrawal, A ratchet-like inter-subunit reorganization of the ribosome during translocation. Nature, 2000. 406: p. 318-322.
57. Gregory, S. T., K. R. Lieberman, and A. E. Dahlberg, Mutations in the peptidyl transferase region of E. coli 23S rRNA affecting translation accuracy. Nucleic Acids Research, 1994.22: p. 279-284.
58. Gregory, S. T. and A. E. Dahlberg, Mutations in the conserved P loop perturb the conformation of two structural elements in the peptidyl transferase center of 23 S ribosomal RNA. Journal of Molecular Biology, 1999. 285: p. 1475-1483.
59. O'Connor, M. and A. E. Dahlberg, Mutations at U2555, a tRNA-protected base in 23S rRNA, affect translational fidelity. Proceedings of the National Academy of Sciences, USA, 1993.90: p. 9214-9218.
60. Kim, D. F. and R. Green, Base-pairing between 23S rRNA and tRNA in the ribosomal A site. Mol.Cell, 1999. 4: p. 859-864.
61. Tapprich, W. E. and A. E. Dahlberg, A single base mutation at position 2661 in E. coli 23S ribosomal RNA affects the binding of ternary complex to the ribosome. Embo J, 1990. 9(8): p. 2649-55.
62. Marchant, A. and M. R. Hartley, Mutational studies on the a-sarcin loop of Escherichia coli 23S ribosomal RNA. European Journal of Biochemistry, 1994. 226: p. 141-147.
63. Gluck, A., Y. Endo, and I. G. Wool, The ribosomal RNA identity elements for ricin and for alpha-sarcin: mutations in the putative CG pair that closes a GAGA tetraloop. Nucleic Acids Research, 1994. 22: p. 321-325.
64. Macbeth, M. R. and I. G. Wool, The phenotype of mutations of G2655 in the sarcin/ricin domain of 23 S ribosomal RNA. Journal of Molecular Biology, 1999.285: p. 965-975.
65. Chan, Y. L., A. S. Sitikov, and I. G. Wool, The phenotype of mutations of the base-pair C2658. G2663 that closes the tetraloop in the sarcin/ricin domain of Escherichia coli 23 S ribosomal RNA. Journal of Molecular Biology, 2000. 298: p. 795-805.
66. Leonov, A. A., et al., Affinity purification of ribosomes with a lethal G2655C mutation in 23S rRNA that affects the translocation. J Biol Chem, 2003.
67. O'Connor, M., et al., Genetic probes of ribosomal RNA function. Biochemistry and Cell Biology, 1995.73: p. 859-868.
68. Sagar, M. B., L. Lucast, and J. A. Doudna, Conserved but nonessential interaction of SRP RNA with translation factor EF-G. Rna, 2004. 10(5): p. 772-8.
69. Nagai, K., et al., NEW EMBO MEMBER'S REVIEW. Structure, function and evolution of the signal recognition particle. Embo J, 2003. 22(14): p. 3479-3485.
70. Green, R., R. R. Samaha, and H. F. Noller, Mutations at nucleotides G2251 and U2585 of 23 S rRNA perturb the peptidyl transferase center of the ribosome. Journal of Molecular Biology, 1997.266: p. 40-50.
71. Samaha, R. R., R. Green, and H. F. Noller, A base pair between tRNA and 23S rRNA in the peptidyl transferase centre of the ribosome. Nature, 1995. 377: p. 309-314.
72. Spahn, C. M. T., et al., Mutational analysis of two highly conserved UGG sequences of 23 S rRNA from Escherichia coli. Journal of Biological Chemistry, 1996. 271: p. 32849-32856.
73. Porse, B. T., H. P. Thi-Ngoc, and R. A. Garrett, The donor substrate site within the peptidyl transferase loop of 23 S rRNA and its putative interactions with the CCA-end of N-blocked aminoacyl-tRNA^{Phe}. Journal of Molecular Biology, 1996. 264: p. 472-483.
74. Thompson, J., et al., Analysis of mutations at residues A2451 and G2447 of 23S rRNA in the peptidyl transferase active site of the 50S ribosomal subunit. Proceedings of the National Academy of Sciences, USA, 2001. 98: p. 9002-9007.
75. Triman, K. L., A. Peister, and R. A. Goel, Expanded versions of the 16S and 23S Ribosomal RNA Mutation Databases (16SMDBexp and 23SMDBexp). Nucleic Acids Research, 1998. 26: p. 280-284.
76. Harms, J., et al., High resolution structure of the large ribosomal subunit from mesophilic eubacterium. Cell, 2001. 107: p. 679-688.
77. Gabashvili, I. S., et al., The polypeptide tunnel system in the ribosome and its gating in erythromycin resistance mutants of L4 and L22. EMBO Journal, 2001. 8: p. 181-188.
78. Chittum, H. S, and W. S. Champney, Ribosomal protein gene sequence changes in erythromycin-resistant mutants of Escherichia coli. J Bacteriol, 1994. 176: p. 6192-6198.
79. Klein, D. J., P. B. Moore, and T. A. Steitz, The roles of ribosomal proteins in the structure assembly, and evolution of the large ribosomal subunit. J Mol Biol, 2004. 340(1): p. 141-77.
80. Nowotny, V. and K. H. Nierhaus, Initiator proteins for the assembly of the 50S subunit from Escherichia coli ribosomes. Proceedings of the National Academy of Sciences, USA, 1982. 79: p. 7238-7242.
81. Dabbs, E. R., A spontaneous mutant of Escherichia coli with protein L24 lacking from the ribosome. Mol Gen Genet, 1982. 187(3): p. 453-8.
82. Cabezon, T., et al., Ribosomal assembly deficiency in an Escherichia coli thermosensitive mutant having an altered L24 ribosomal protein. J Mol Biol, 1977. 116(3): p. 361-74.
83. Diedrich, G., et al., Ribosomal protein L2 is involved in the association of the ribosomal subunits, tRNA binding to A and P sites and peptidyl transfer. EMBO Journal, 2000.19: p. 5241-5250.
84. Khaitovich, P., et al., Reconstitution of functionally active Thermus aquaticus large ribosomal subunits with in vitro-transcribed rRNA. Biochemistry, 1999. 38: p. 1780-1788.
85. Kramer, G., et al., L23 protein functions as a chaperone docking site on the ribosome. Nature, 2002. 419(6903): p. 171-4.
86. Jenni, S. and N. Ban, The chemistry of protein synthesis and voyage through the ribosomal tunnel. Curr Opin Struct Biol, 2003. 13(2): p. 212-9.
87. Pool, M. R., et al., Distinct modes of signal recognition particle interaction with the ribosome. Science, 2002. 297(5585): p. 1345-8.
88. Sergiev, P. V., et al., The conserved A-site finger of the 23S rRNA. just one of the inter subunit bridges or apart of the allosteric communication pathway? J Mol Biol, 2005.
353(1): p. 116-23.

1.-10. (canceled)

11. A method of identifying an antibiotic target site in a ribosome comprising:
(a) providing a plurality of mutant ribosomal RNA (rRNA) genes;
(b) screening the plurality for a deleterious rRNA mutation; and
(c) mapping the deleterious rRNA mutation,
wherein an antibiotic target site is identified by a deleterious rRNA mutation.

12. The method of claim 11, wherein the plurality of mutant rRNA genes is enriched.

13. The method of claim 12, wherein the plurality of mutant rRNA genes is enriched by negative selection.

14. The method of claim 11, wherein the rRNA is from a small ribosomal subunit.

15. The method of claim 11, wherein the rRNA is from a large ribosomal subunit.

16. The method of claim 11, wherein the rRNA gene is from a microorganism.

17. The method of claim 16, wherein the microorganism is a bacterium.

18. The method of claim 16, wherein the microorganism is pathogenic to a mammal.

19. An antibiotic target site identified by the method of claim 11.

20. An antibiotic target site, wherein the target site is selected from a site listed in Table 1.

21. The antibiotic target site of claim 20, wherein the target site comprises two or more sites listed in Table 1.

22. The antibiotic target site of claim 21, wherein cluster I, cluster II, cluster III, or cluster IV comprises the sites.

23. An antibiotic target site, wherein the target site is selected from a site listed in Table 2.

24. The antibiotic target site of claim 23, wherein the target site comprises two or more sites listed in Table 1.

25. The antibiotic target site of claim 24, wherein cluster V, cluster VI, cluster VII, cluster VIII, or cluster IV comprises the sites.

26. A method for identifying an antibiotic comprising:
(a) identifying a compound that binds to a target site according to any one of claims 19-25.
